(19) United States
(12) Patent Application Publication
(10) Pub. No.: US 2007/0054337 A1
(43) Pub. Date: Mar. 8, 2007

(54) Nanoparticle conjugates and method of production thereof

(76) Inventors: David Garth Ferning, Liverpool (GB); Dguyen Thi Kim Thanh, Liverpool (GB); John Arthur Smith, Liverpool (GB); Christopher Richard Doty, Ann Arbor, MI (US); Raphael Levy, Liverpool (GB)

Correspondence Address: MORRISON & FOERSTER LLP, 12531 HIGH BLUFF DRIVE, SUITE 100, SAN DIEGO, CA, 92130-2040, US

(21) Appl. No.: 10/572,435

(22) PCT Filed: Sep. 20, 2004
(86) PCT No.: PCT/GB04/03986

§ 371 Date: Aug. 23, 2006

(30) Foreign Application Priority Data
Sep. 19, 2003 (GB) 0321937.5

(51) Int. Cl. G01N 33/551

(52) U.S. Cl. 435/7.92; 436/524; 977/902

(57) ABSTRACT

The present invention relates to a nanoparticle conjugate comprising a nanoparticle conjugated to a plurality of peptides of a substantially similar amino acid sequence, a peptide conjugated to the nanoparticle by means of a Cysteine (C) residue and the nanoparticle conjugate further comprising a ligand attached to the peptide and additionally an identification means. The present invention also relates to method of producing the nanoparticle conjugate.

The present invention relates to a nanoparticle conjugate comprising a nanoparticle conjugated to a plurality of peptides so as to form a peptide shell which stabilises the nanoparticles and allows stoichiometric coupling of biomolecules, the peptide further comprising a ligand.

The use of nanoparticles in the field of life sciences has not been as wide spread as originally expected, despite the development of this technology over recent years. Although nanoparticles have a wide potential application range, their use as diagnostic or therapeutic agents are associated with a number of problems. For example, maintaining the stability of conjugates adhered to nanoparticles in conditions similar to that found in cellular tissue and/or fluids has posed a number of problems due to the diverse chemical environment. Additionally problems regarding sensitivity of detecting the nanoparticles are also experienced.

Surface plasmons in metallic nanoparticles (collective oscillations of electrons confined within the particle) have a characteristic resonance frequency, depending on particle size. Thus, white non-polarised light causes metallic nanoparticles to emit light at a wavelength characteristic of their size in a phenomenon known as resonant light scattering (RLS) (Elghanian, R., et al. (1997) Science, 277: 1078-1081 and Schultz, S. et al., (2000) Proc. Natl. Acad. Sci. USA, 97: 996-1001). The wavelength of scattered light depends on the nanoparticle size and the medium surrounding it. Importantly, both their electronic and optical properties depend in a sensitive manner on their environment. and these properties can be used as extremely sensitive probes (Elghanian, R., et al. (1997); Schultz, S. et al., (2000) and Baum, T. et al., (1999) Langmuir, 15: 866-871). Three techniques have been developed to detect metallic nanoparticles at the single particle level. Surface plasmons in metallic nanoparticles (collective oscillations of electron confined within the particle) have a characteristic resonance frequency, depending on particle size. Thus, nanoparticles absorb, emit and scatter light in a manner characteristic of their size. The three techniques are as follows: (i) Resonant light scattering (RLS) by single particles is readily detected by simple darkfield microscopy if these are fairly large (30-40 nm Ag, 50-60 nm Au), but is extremely difficult with samples, e.g., cells, conventional glass slides, that scatter strongly (Doty, C., et al (2004) Cell. Mol. Life Sci. (61), 1843-1849); (ii) RLS from an evanescent field at a surface reduces the size of particle that can be detected by ˜4-fold and reduces the problem of background scattering; and (iii) Photothermal detection, developed by the nanophotonic laboratory in Bordeaux can detect particles as small as 2 nm without the problem of background scattering (Boyer, D., et al., (2002) Science, (297), 1160-1163 and Cognet, L., et al (2003) Proc. Natl. Acad. Sci. USA, (100), 11350-11355).

Significantly, the signal output from metallic nanoparticles on a molar basis is far greater (10³ to 10⁶ -fold) than that of the fluorophores conventionally used in biology, which are subject o quenching and the signal output is not prone blinking, as found with Q-dots (Doty, C., et al. (2004)). In addition, bringing two such particles in close proximity results in a change in the signal output (due to plasmon coupling) as a shift of wavelength of scattered light in the case of RLS detection, or an increase in the amplitude of signal in the other detection systems. Thus, a sensing mechanism for biomolecular interactions based simply on tagging complementary biomolecules with nanoparticles is readily accessible, e.g., Elghanian, R., et al. (1997)). Nanoparticles are introduced into cells using standard transfection methods. Therefore metallic nanoparticles offer a unique route to simple probes for biomolecular function with a sensitivity of a single particle.

Similar considerations have been given to the fluorescent properties of semiconductor nanoparticles that are commonly called “Q-dots”. These exhibit a narrow fluorescence emission dependent on particle size and are not prone to “photobleaching”. Finally, the optical range of nanoparticles is such that a high degree of multiplexing is possible.

Given the above remarkable properties (which should revolutionise fields such as diagnostics, including over-the-counter (OTC) home diagnostics for tests currently performed on blood samples, and biomolecular research, including High Throughput Screening (HTS)), it is surprising that metallic and semiconductor nanoparticles have had little impact in the real world. One of the reasons why metallic and semiconductor nanoparticles have not been that successful to date is due to the signal-to-noise ratios. Whereas under ideal physical conditions, e.g., organic solvents or pure water, nanoparticles afford simple single particle detection, their propensity to aggregate in solutions containing electrolyes and to bind virtually all macromolecules non-specifically drastically reduces the sensitivity of assays based on nanoparticles. Current approaches to reduce the ‘stickiness’ (aggregation of nanoparticles to one another) of nanoparticles involve building a ligand shell around the nanoparticles. The ideal ligand shell protects the nanoparticles from the complex environment of biological systems and provides the means to specifically couple at a defined stoichiometry functional biological molecules of a defined thickness, to allow assays to take advantage of the coupling effect observed when nanoparticles are in close proximity.

Since thiols form strong covalent bonds with nanoparticles, existing ligand shells often possess one or more thiols and include alkyl thiols and derivatives, e.g., mercaptoundecanoic acid (MUA), lipoate, thiolated dextrans and polyethylene glycols. The simple ligand shells that produce a thin self-assembled monolayer, e.g., MUA, lipoate, cysteine, glutathione (ECG), are attractive since they provide a defined chemical environment and the thickness of the ligand shell is controlled by the length of the monolayer units. However, in the best cases these ligand shells provide only a partial stabilisation in aqueous biological solutions. The more complex polymers, e.g., thiolated dextrans and polyethylene glycols, do produce reasonably stable nanoparticles. However, the thickness of these ligand shells cannot be controlled and the polymers are known to form local microenvironments that can adsorb biological macromolecules and stoichiometric coupling of macromolecules is difficult and often impossible.

It is therefore an object of the present invention to provide a nanoparticle conjugate comprising a peptide capping ligand that stabilises the nanoparticle and may also allow stoichiometric coupling of biomolecules. It is also an object of the present invention to provide a method of producing the nanoparticle conjugates.

In accordance with the present invention, there is provided a nanoparticle conjugate comprising a nanoparticle conjugated to a plurality of peptides of a substantially similar amino acid sequence, the peptide conjugated to the nanoparticle by means of a Cysteine (C) residue and the nanoparticle conjugate further comprising a ligand attached to the peptide.

Therefore, the present invention provides for a nanoparticle conjugate that has a greatly increased stability in a number of biological and chemical environments. The configuration of the nanoparticle conjugate resembles a protein which may additionally have a “sticky’ core (containing for example, an inorganic metallic or semiconductor material) that is hidden by an organised surface (provided by the peptide) that can therefore be tailored to suit the needs of a given application. It is believed that the peptide secondary structure (alpha helix, beta strand, H-bonding) assists in nanoparticle stabilisation. Indeed, peptides that form beta strands are preferred as the strand formation allows high packing densities of peptides to nanoparticles to be achieved.

Preferably, the Cysteine (C) residue is conjugated to the nanoparticle by means of its thiol and the amino group. Furthermore, it is preferable that the distal end of the peptide terminates with a carboxyl group or that the peptide is conjugated to the ligand by means of a carboxyl group. The exact choice of amino acid sequence will be governed by amino acids that allow close packing on the nanoparticle surface and this in turn will be dictated by the curvature of the nanoparticle amongst other things. The core peptide may have the general sequence of CXn(ligand), CCXn(ligand), CXn(ligand)Xn or or CCXn(ligand)Xn, where X denotes any amino acid residue and n denotes any length of amino acid residues. Preferably, the peptide sequence independent of the ligand has the sequence H₂N-Cysteine-Alanine-Leucine-Asparagine-Asparagine-COOH (CALNN) or H₂N-Cysteine-Cysteine-Alanine-Leucine-Asparagine-Asparagine-COOH (CCALNN).

In accordance with another aspect of the present invention, there is provided a nanoparticle conjugate comprising a nanoparticle conjugated to a plurality of peptides of a substantially similar amino acid sequence, the peptide conjugated to the nanoparticle by means of a Cysteine (C) residue located in a central region of the peptide.

Should a peptide be conjugated to the nanoparticle by means of centrally located Cysteine residues, there may be two or more Cysteine residues located in a central region of the peptide. Preferably, the peptide sequence either side of the Cysteine residues are substantially symmetrical. The peptide may therefore have the general sequence of (ligand)XnCXn(ligand), Xn(ligand)XnCXn(ligand)Xn, (ligand)XnCCXn(ligand) or Xn(ligand)XnCCXn(ligand)Xn where X denotes any amino acid residue and n denotes any length of amino acid residues. Preferably, the peptide independent of the ligand has the sequence NNLACALNN or NNLACCALNN.

The nanoparticle conjugates may further comprise an identification means attached to the peptide or the ligand. The nanoparticle conjugate may further comprise a functional group (in addition to or not to an identification means) attached to the peptide or ligand. An “identification means” should be taken to include functional groups also. An additional sequence of amino acid residues is disposed between the ligand and the identification means and/or functionalised group and/or the ligand or identification means or functional group. Therefore, a “spacer” element may be placed between the core peptide sequence and the ligand and/or placed between the ligand and the identification means/functional group. Furthermore, an additional sequence of amino acid residues may be disposed after the ligand if no identification means/functional group is present, or alternately, the residues may be disposed after the identification means if it is present. The additional sequence may comprise any sequence, but preferably, it comprises two or more Glycine residues.

The nanoparticle conjugate may comprise different subgroups of peptides. Additionally, different ligands and optionally, different identification means and/or functional groups may be attached to different subgroups of peptides. Therefore a single nanoparticle (or small number of nanoparticles) may be produced with a range of ligands and/or identification means and/or functional groups. The single nanoparticles can then be used in conjunction with others of a known activity to perform multiple testing of samples. Indeed, a nanoparticle conjugate may have a mixture of peptides having ligands with additional amino acid residues at the distal end of the peptide and ligands with no additional amino acid residues. It will be apparent that this will assist greatly in a number of experiments, for example the co-localisation of two or more proteins in a sample.

The nanoparticle may be produced from a number of materials which will be apparent to one skilled in the art. Preferably, the nanoparticle is produced from one of the following materials; a metallic material, a magnetic material or a semi-conducting material. Such materials may be gold, silver, cobalt, nickel, platinum, cadmium selenide or zinc sulphide (or other materials used to produce “quantum dots” or similar particles).

Magnetic nanoparticles have many applications in biomedicine, such as contrast enhancement agents for magnetic resonance imaging, targeted therapeutic drug delivery and hyperthermia treatment for cancers (Berry, C. C. and Curtis A. S. G., (2003) J. Phys. D: Appl. Phys. 36: R189-206 and Parkhurst, Q. A. et al., (2003) J. Phys. D: Appl. Phys. 36: R167-181). Magnetic immunoassay techniques have also been developed in which the magnetic field generated by the magnetically labeled targets is detected directly with a sensitive magnetometer (Chemla, Y. R., et al., (2000) P. Natl. Acad. Sci. USA. 97: 14268-14272) and such techniques may be used in accordance with the present invention.

It will be apparent to one skilled in the art that should magnetic nanoparticles be employed in accordance with the present invention, such nanoparticles will preferably possess large saturation magnetisation and high magnetic susceptibility so that they respond strongly (Sensitive) to small external/applied magnetic fields or the signal of a magnetic sensor; but weakly respond to other forces such as gravity, Brownian motion, viscosity, van der Waals interactions. Furthermore, the nanoparticles may also be superparamagnetic at room temperature (i.e. the magnetic moment fluctuates freely in the absence of a magnetic field and thus it behaves as non-magnetic) so as to avoid the aggregation of particles. The full exploitation of these properties of magnetic nanoparticles may require size or shape monodispersity and complete or substantially complete stability in biological environments, including, stability in air and aqueous solutions.

The identification means may be selected from a number of molecules and/or compounds that are commonly used for identifying or “tagging” the binding of a ligand to a target molecule. It will be appreciated that molecules and compounds which have yet to be developed may also be employed as an identification means. Preferably, the identification means and/or functionalised group and/or ligand is selected from one or more of the following: biotin and/or avidin, streptavidin, streptactin, Histidine tags, NTA, radio active labels, antigens, epitopes or parts of epitopes, antibodies, fluorochromes, nucleic acids, recognition sequences, enzymes, antibodies, peptides, proteins, receptors or a target molecules, saccharides, polysaccharides and lipids. The identification means and/or functional group may comprise heparnin sulphate and such a nanoparticle conjugate may be conjugated with a mercury adduct.

The use of synthetic peptides as ligand shells (peptides with identification means and/or functional groups attached thereto) uniquely allows stoichiometric derivatisation by incubating nanoparticles with a defined ratio of native peptide and peptide with an extension which acts as a tag/synthon or is a recombinant protein. Purification, if necessary can therefore make use of standard chromatographic properties of the tag/synthon or coupled macromolecule.

It follows that synthetic peptide chemistry, which is automated and extremely versatile, can be used to introduce identification means and/or functional groups (such as tags) into the peptides. The identification means and/or functional groups need not be natural and may be unnatural (the latter including D-amino acids and amino acids with synthetic side chains possessing unique chemical reactivities, for example).

The ligand conjugated to the nanoparticle may be a number of different molecules that are capable of binding with other molecules in order to either adhere the nanoparticle to a particular site which may be for identification of a certain molecule within a sample or to hold a molecule for later purification. Furthermore, the ligand may also be used to direct the nanoparticle to a certain site, for example to a cell expressing a certain epitope in order to deliver a pharmaceutical compound. Preferably, the ligand may be selected from one or more of the following: nucleic acid, an antibody, a peptide, a protein, a receptor or a target molecule, a saccharide, a polysaccharide and a lipid.

The nanoparticle conjugate may also be capable of being conjugated to at least one other nanoparticle conjugate or is conjugated to at least one other nanoparticle conjugate so as to to form nanoparticle conjugate assemblies. Such assemblies can be used for probing or diagnostic tools for identifying a number of variables, such as a number of different antigens on a cell surface, or as a means to amplify the signal by increasing the number of nanoparticles associated with a primary nanoparticle-analyte interaction, for example.

The nanoparticle conjugate as described herein above may additionally have a compound or part of a compound of a pharmaceutically active salt conjugated to the nanoparticle or attached to the peptide. Therefore the delivery of therapeutic compounds can be directed to different cells or cytological constituents. The provision of part of a pharmaceutically active salt may allow the two-step approach of pro-drug therapy to be utilised.

The nanoparticle may have a diameter in the range of 1-100 nm. Preferably, the plurality of peptides may substantially cover the surface of the nanoparticle so as to provide a shell around the nanoparticle. It will therefore be apparent to one skilled in the art that such a shell will “shield” the nanoparticle core throughout a number of cytological and biological environments and allow the nanoparticle to remain extremely stable. The shell permits optical detection of the nanoparticle including coupling effects for multiple nanoparticles.

A 12.3 nm gold nanoparticle may have up to approximately 855±70 peptides per nanoparticle, although potentially this figure could be within the range of 500-1500 peptides per nanoparticle. On this basis a substantially spherical nanoparticle with a diameter of 12.3 nm would have an approximate surface area of 475 nm², which equates to allowing between approximately 1.1-3.2 peptides per nm² of the nanoparticle, although the number of peptides can be tailored for different applications and it will also be dependent not only upon the total surface area of the nanoparticle but also its curvature. It is preferred that there are approximately in the range of 1-3.2 peptides per per nm² of the nanoparticle. Furthermore, the density of peptides on a nanoparticle may differ for larger nanoparticles. Preferably, the density of peptides per nanoparticle will be as high as possible in order to obtain a close packed arrangement.

The nanoparticle conjugate may be used in producing diagnostic assays, separating and/or purifying proteins or producing therapeutic agents. It will be evident that the nanoparticle conjugate can be used in a wide range of applications within biology and chemistry and allied fields. Preferably, the nanoparticle conjugate may be used in conjunction with any of the following techniques: chromatography, Enzyme linked immunosorbent assay (ELISA), lyophilisation, Fluorescence in situ hybridisation (FISH), in situ hybridisation (ISH), SDS-polyacrylamide gel electrophoresis (PAGE), flow cytometry, immunohistochemistry, protein purification, western blotting, cytogenetic analysis, molecular interaction assays, histochemistry on fixed and living cells/tissue and high throughput screening; “bottom up” assembly of nanoparticles directed by the tags/ligands for construction of nanodevices, including cases where the peptide shell and/or the tags/ligands are subsequently removed.

In accordance with another aspect of the present invention, there is provided a method of producing a nanoparticle conjugate as herein described above by incubating in water, nanoparticles stabilised in a stabilising solution with a plurality of peptides in a phosphate buffered saline. Such as stabilising solution will be evident to the skilled addressee and may comprise a citrate, acrylate or oxalate solution and others yet to be developed. The one or more ligands and optionally one or more identification means and/or functional groups may be conjugated to the peptide prior to incubation with the nanoparticle or during the course of the incubation. For 12.3 nm Au nanoparticles, the peptide will commonly be dissolved in PBS, pH 7.2 and incubated with citrate-stabilised nanoparticles (6 nM) with peptide (1 mg/ml to 10 μg/ml) (1 vol to 1 vol).

In accordance with a yet another aspect of the present invention, there is provided a method of producing a nanoparticle conjugate as herein described above by including a plurality of peptides comprising one or more ligands and optionally one or more identification means during the synthesis of the nanoparticle.

Both methods of producing a nanoparticle conjugate may additionally employ the use of freeze drying so that the nanoparticle conjugate can be stored or transported prior to use. It will be apparent that this may be required for certain ligands that may degrade or denature over time.

The nanoparticle conjugates can be used as molecular interaction sensors. The “colour” of the nanoparticles depends on their size and nanoparticle size may be changed by simply bringing two or more particles into close association (at the nm scale, so that it is representative of the protein scale) such that their dipoles couple. Nanoparticle conjugates incorporating identification means and/or functional groups can therefore be used as molecular interaction sensors, such as a receptor dimerisation sensor. Such sensors would be highly efficient (high sensitivity, no background, low amounts of macromolecules required) in high throughput screening applications in order to search for compounds whose activity is exerted by preventing or enhancing a molecular interaction. Such sensors would also allow highly efficient detection of a molecule(s) that causes dimerisation or oligomerisation of the “receptors”

The nanoparticle conjugates may be used for analysis of complex secondary gene products. For example, glycomics is an area which suffers from the fact that synthesis is not template driven. Therefore analytical tools and assays are only as good as purification methods and the sensitivity of detection systems. Nanoparticle conjugates with a saccharide binding function, e.g., hydrazide for reducing sugars, would increase sensitivity by several orders of magnitude. Users employing this method would be research laboratories utilising screening assays, etc.

The nanoparticle conjugates may also be used in bioelectronics applications, which so far have up until now been largely confined to using DNA as the scaffold. The interactions from any bioassay in can be used in bioelectronic device assembly [Simon -not sure if I have retained the meaning you were aiming for??). Moreover, many such interactions lend themselves to switching. One example would be coupling a nanoparticle to a redox group or protein, e.g., azurin, to form an actuator. Further examples may include phosphorylation-dephosphorylation and Ca²⁺-induced conformation changes and consequent binding reactions. In some cases the organic material may be partially or completely removed, sometimes by means that fuse the nanoparticles to exploit the structures or linkages between the nanoparticles afforded by the tags on the peptide shell. In short, by virtue of the specificity and range of the tags, which may be placed on the peptide shell, the range of combinatorial ordered assemblies available to bring together nanoparticles augments considerably the applications in bioelectronics.

The present invention will now be more particularly described with reference to the following example and figures.

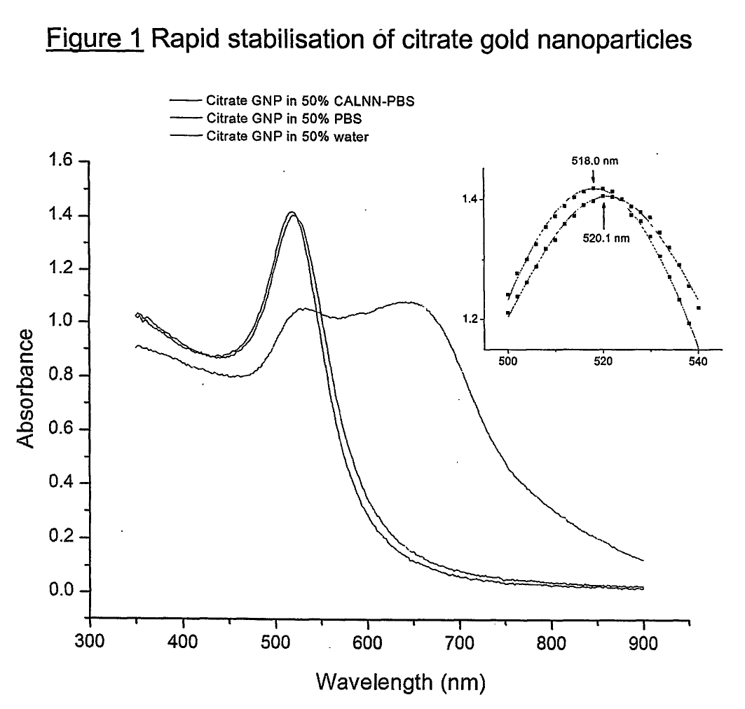

FIG. 1 is a graphical representation of the results of the spectrum analysis at 200-800 nm of citrate Au nanoparticles and CALNN Au nanoparticles as described in Example 1;

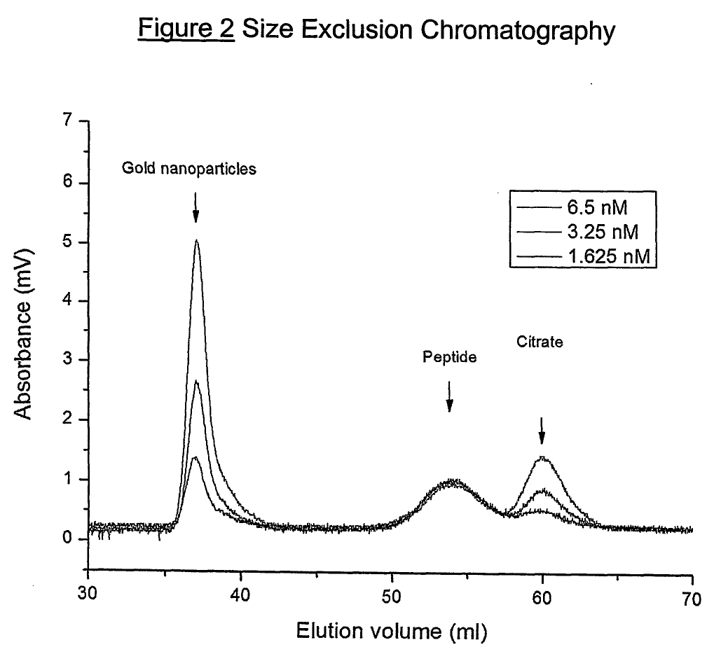

FIG. 2 is a graphical representation of the results of a Sephadex G25 size-exclusion chromatographic purification of CALNN Au nanoparticles from peptide and citrate as described in Example 2;

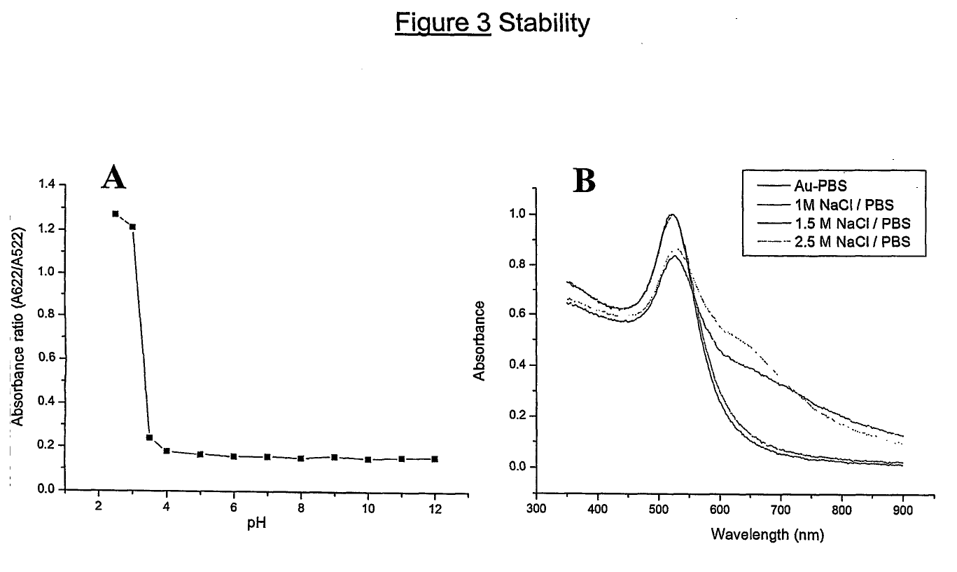

FIG. 3 is a graphical representation of the results from stability experiments of the nanoparticle conjugates over a range of pH (A) and NaCl concentration (B) as described in Example 3;

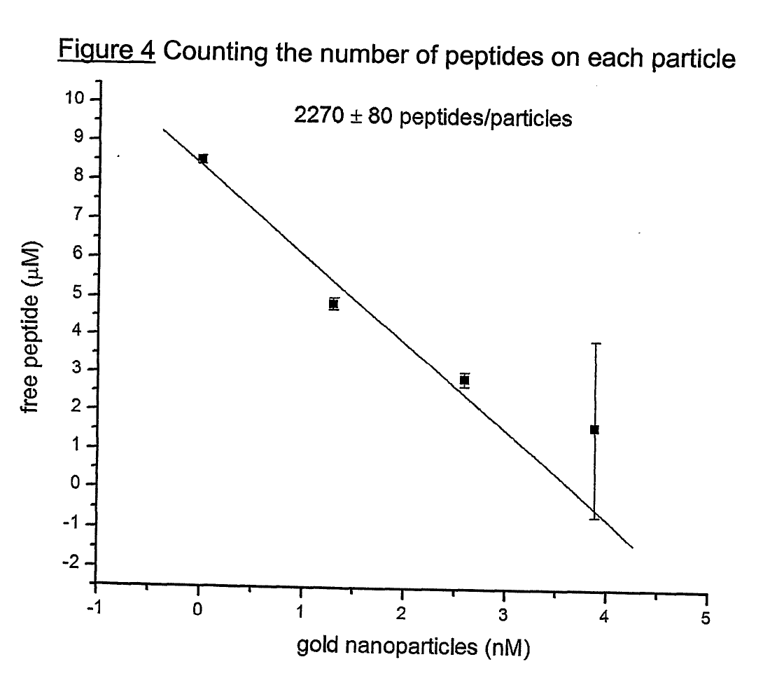

FIG. 4 is a graphical representation of the results of a quantification experiment of the adsorbed peptide by titration as described in Example 4;

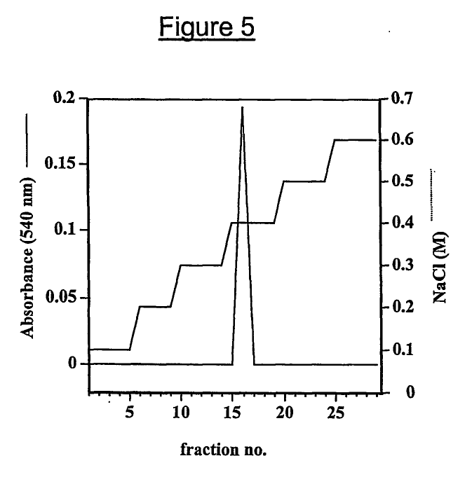

FIG. 5 is a graphical representation of an orientation experiment of peptide on Au nanoparticles by anion-exchange chromatography on DEAE Sepharose fast flow as described in Example 5;

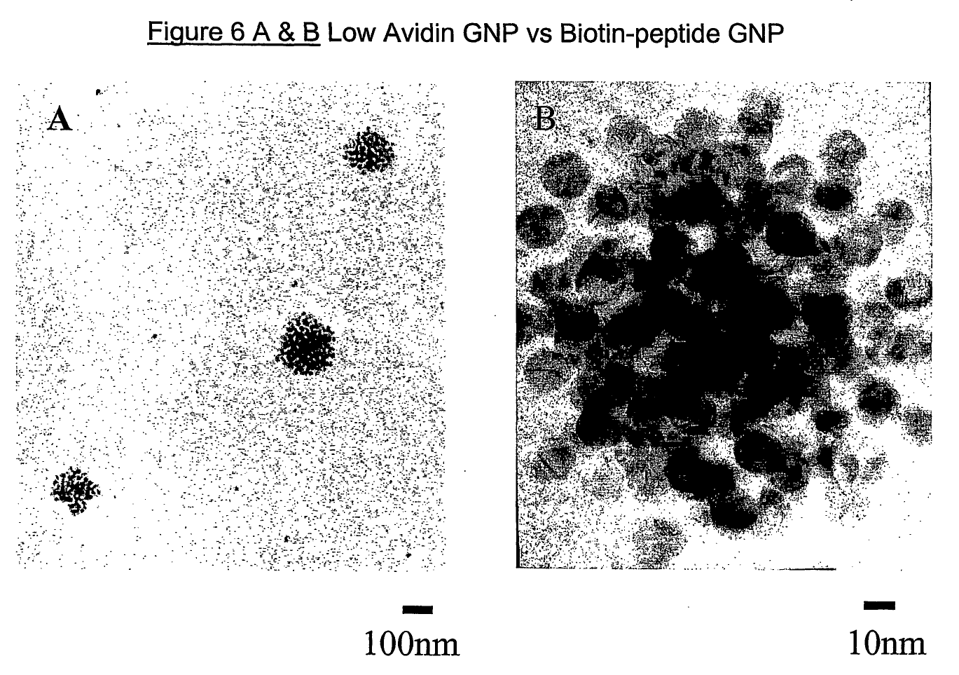

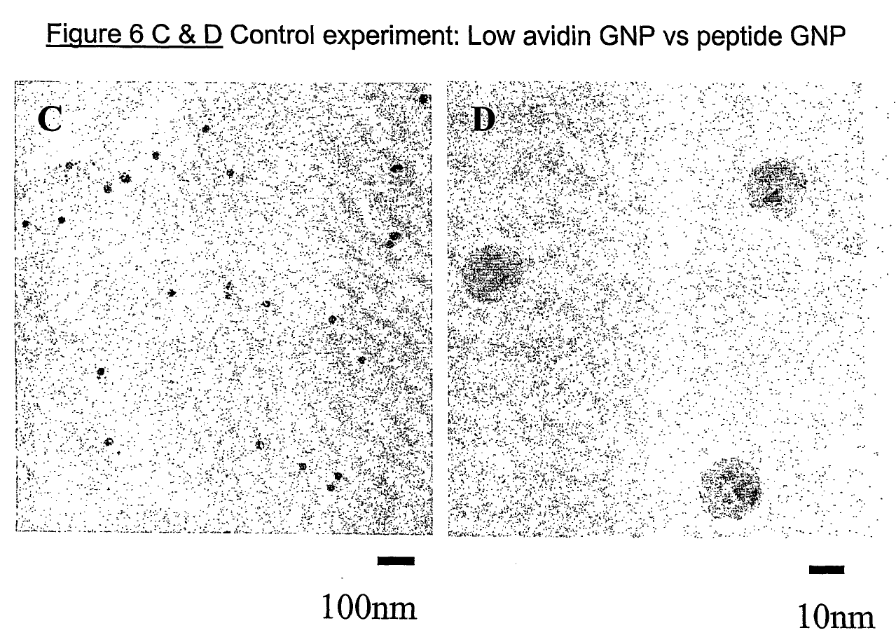

FIG. 6 (A to F) are micrographs showing the specific association of nanoparticles driven by ligands on the peptide by the addition of biotinylated peptide to the ligand shell and subsequent adsorption of nanoparticles to streptavidin-coated nanoparticles as described in Example 6;

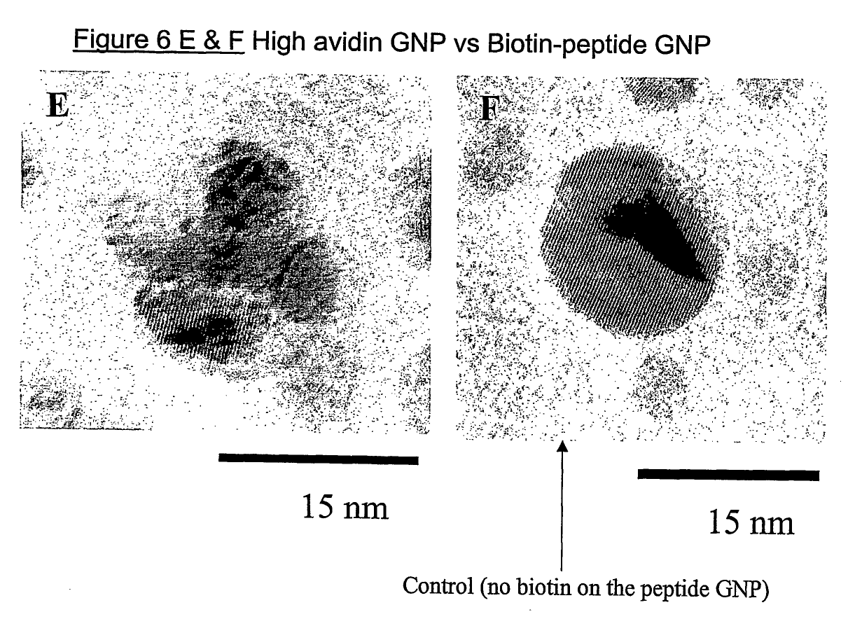

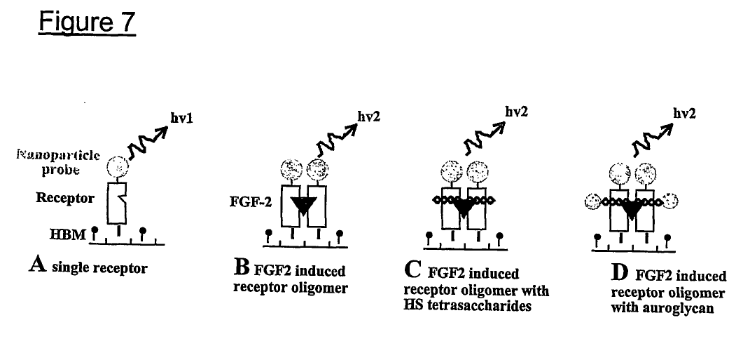

FIG. 7 shows diagrams of examples of a nanoparticle-based sensor for ligand-induced dimerisation in accordance with the present invention;

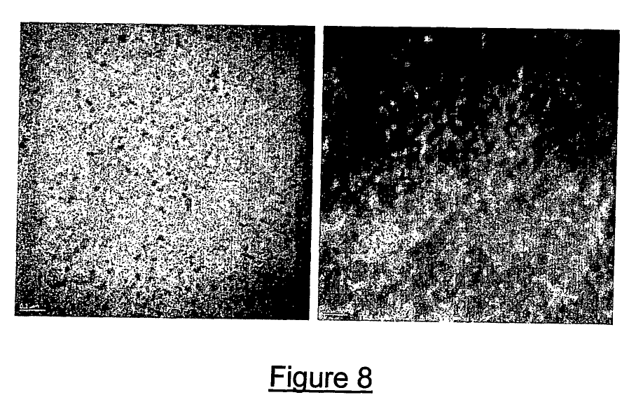

FIG. 8 is transmission electron microscopic images of Co nanoparticles synthesised with a peptide (bright field (left) and dark field (right)) in Example 10;(examples 7, 8, 9?)

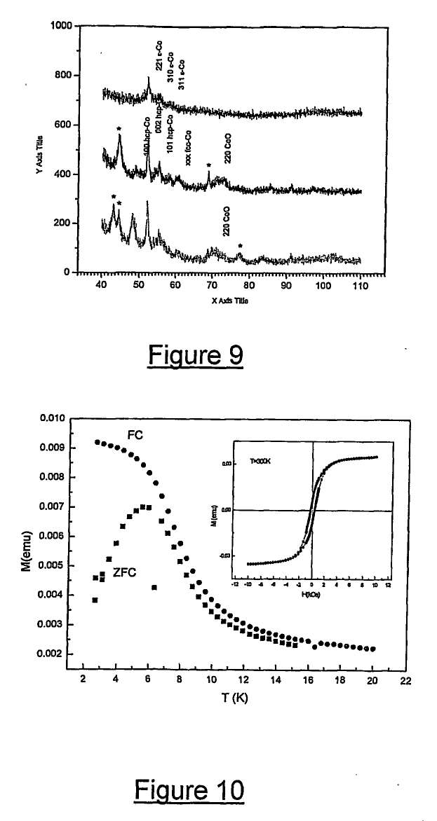

FIG. 9 is an X-ray diffraction characterisation data for the nanoparticles in Example 10;

FIG. 10 shows SQUID characterisation data for the nanoparticles in Example 10;

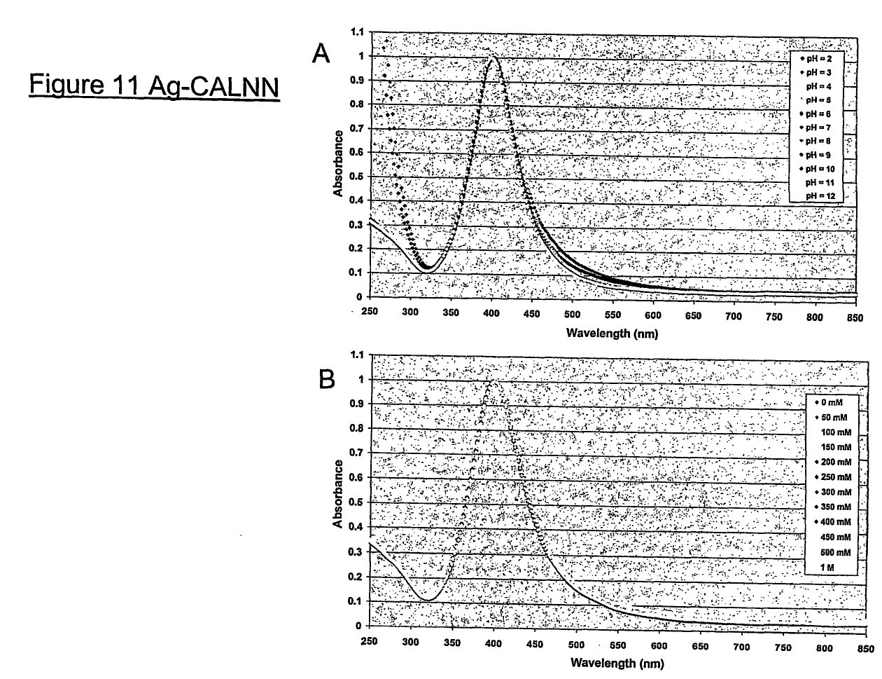

FIG. 11 shows the results of an Ag-CALNN nanoparticle conjugate as produced in Example 11;

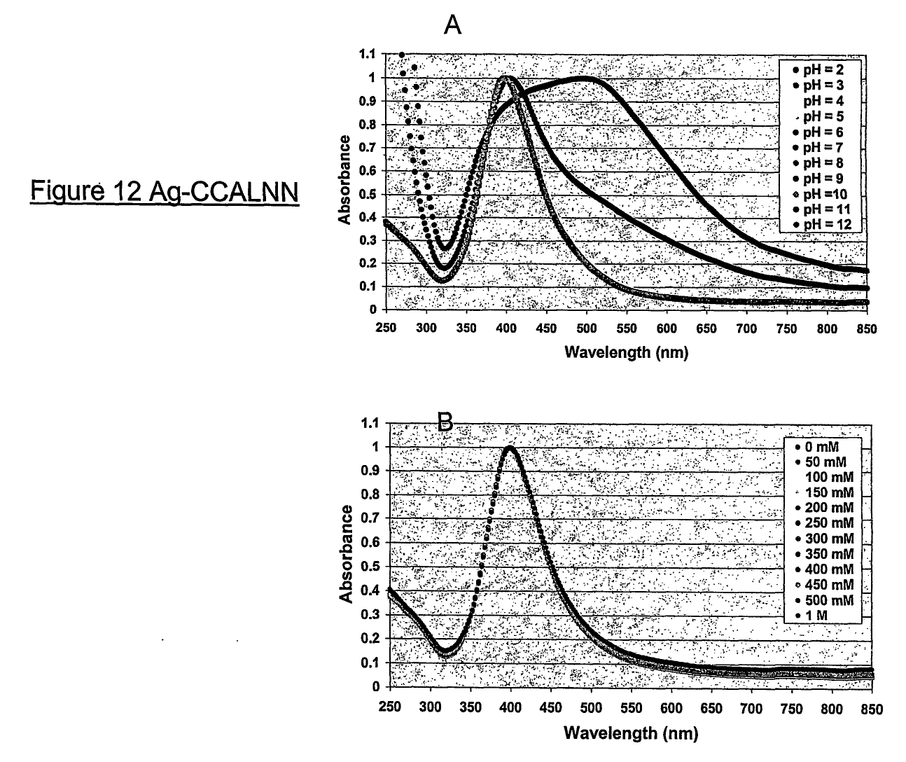

FIG. 12 shows the results of an Ag-CCALNN nanoparticle conjugate as produced in Example 11;

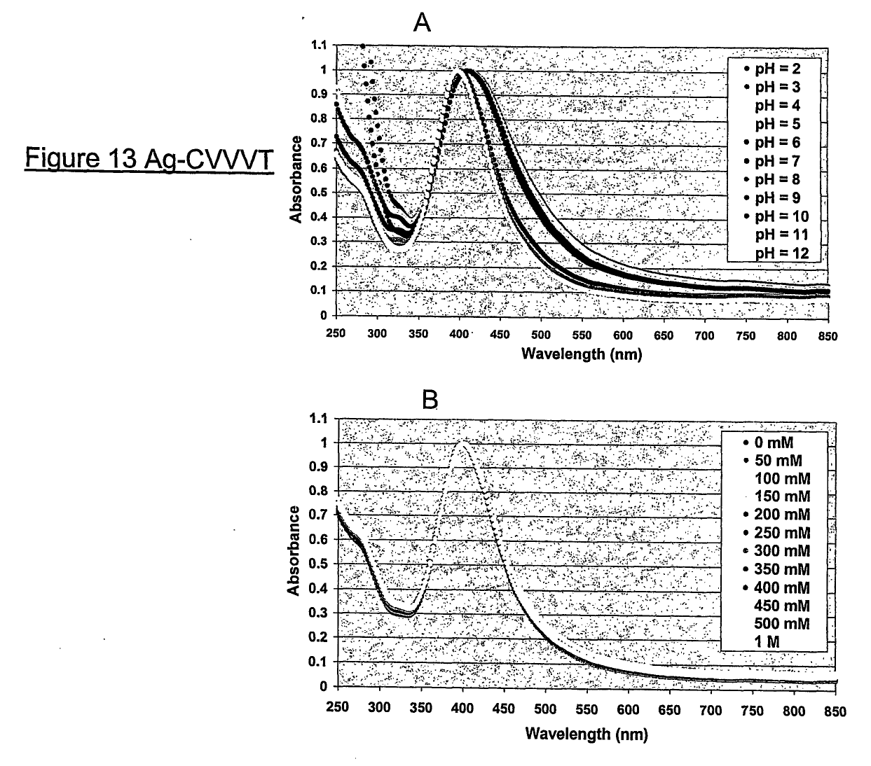

FIG. 13 shows the results of an Ag-CVVVT nanoparticle conjugate as produced in Example 11;

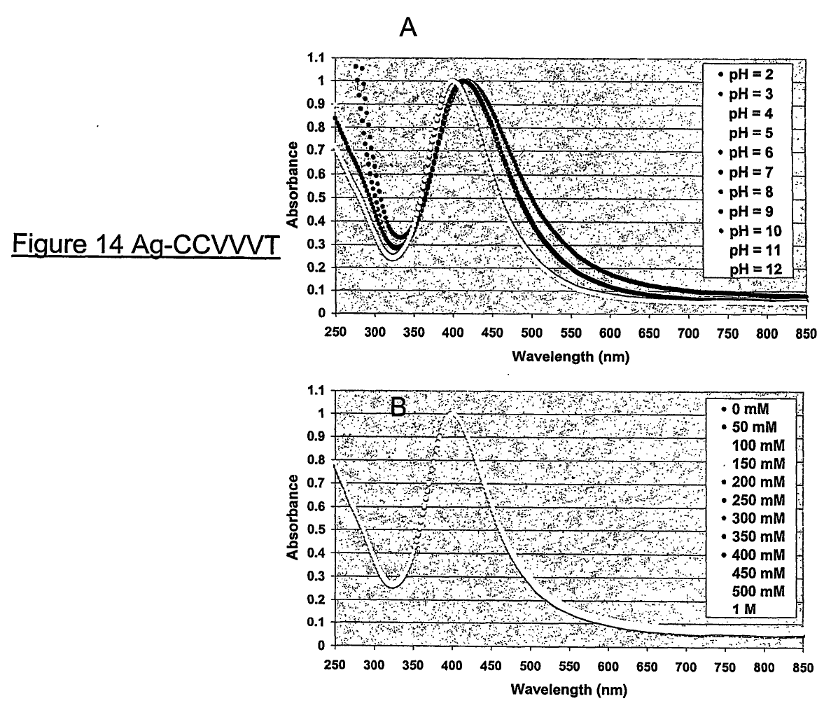

FIG. 14 shows the results of an Ag-CCVVVT nanoparticle conjugate as produced in Example 11;

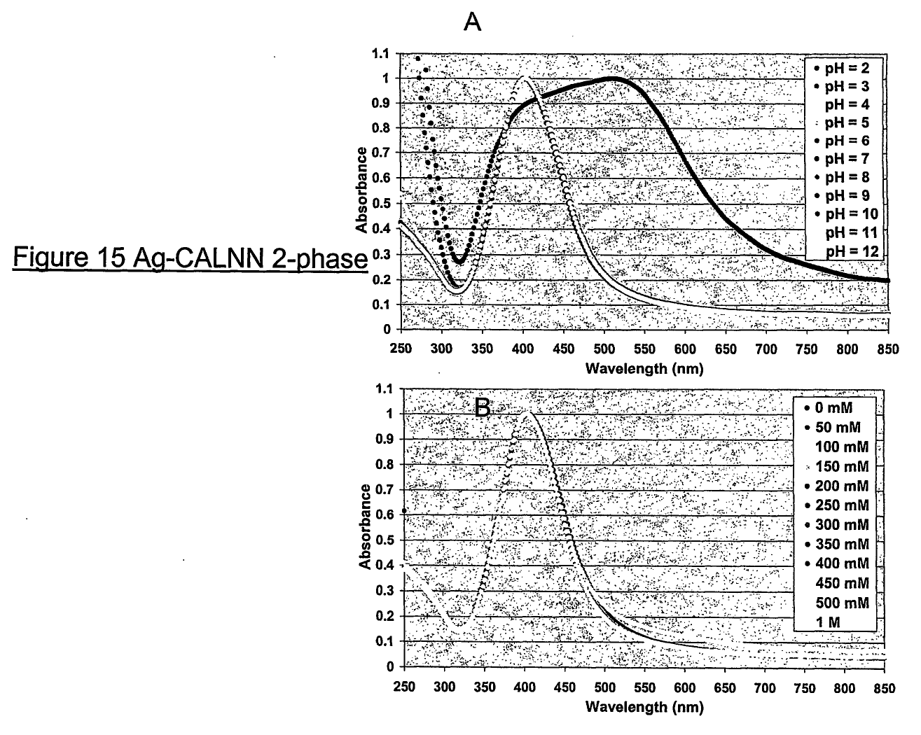

FIG. 15 shows the results of an Ag-CALNN nanoparticle conjugate as produced in Example 11;(appears to be a repeat text of FIG. 11?)

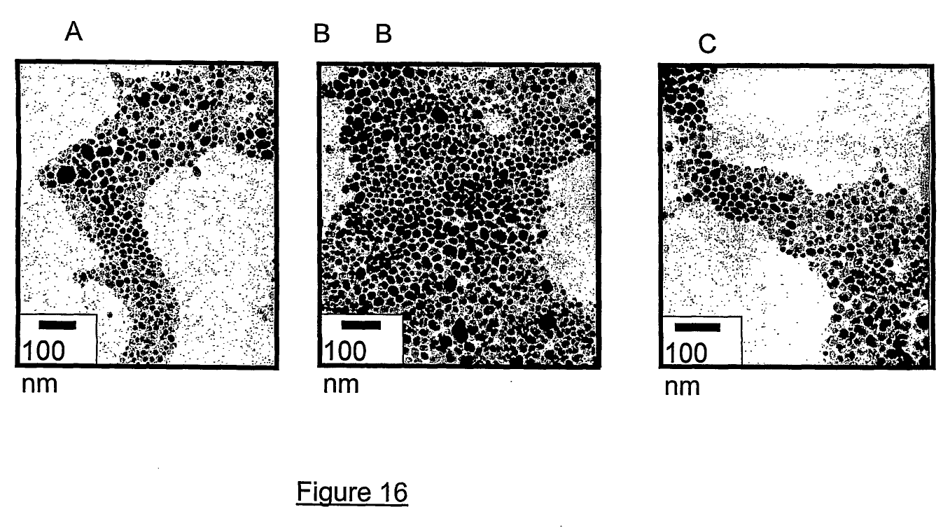

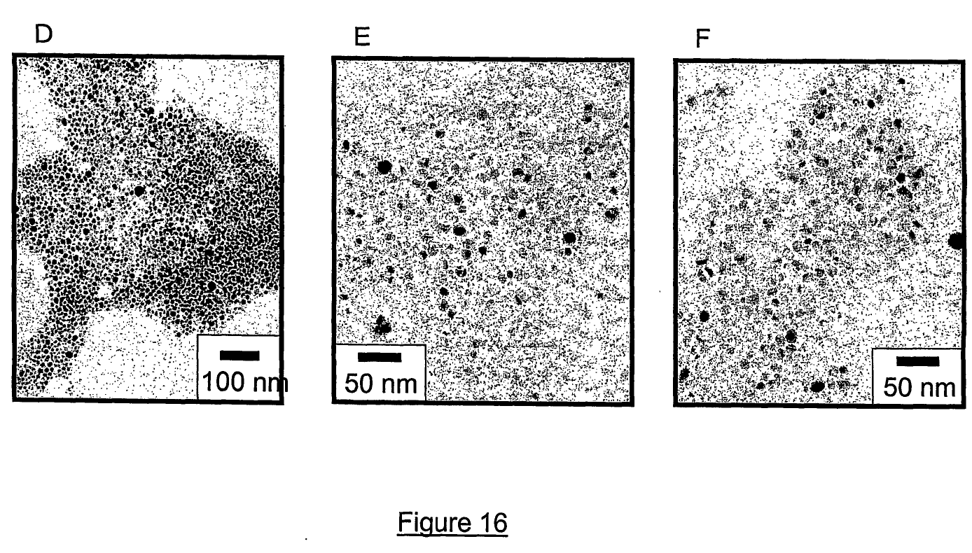

FIG. 16 shows an electron microscope image of Ag nanoparticle conjugates as produced in Example 11;

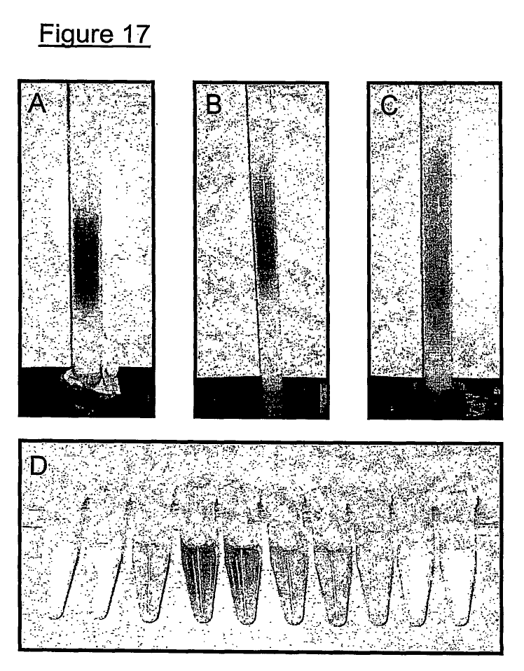

FIG. 17 are photographs of the results of the separation of Ag-PEG and Au-CCALNN on a CL-6B column as produced in Example 11;

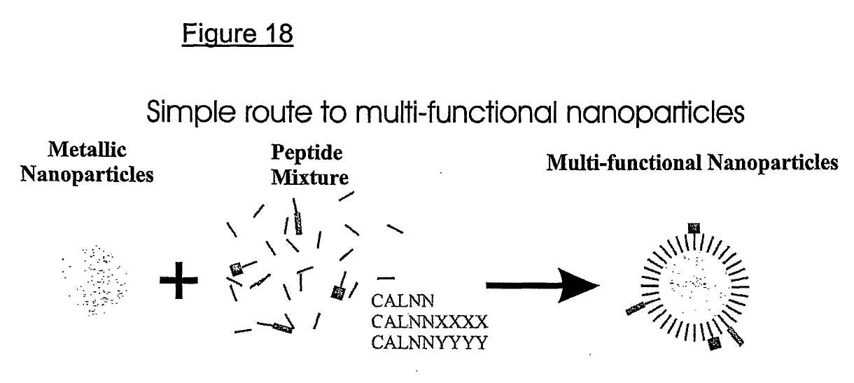

FIG. 18 is a schematic representation of the principle of using a matrix peptide (CALNN in this case) for stabilisation and a defined mole percentage of one or more peptide species as illustrated in Example 12;

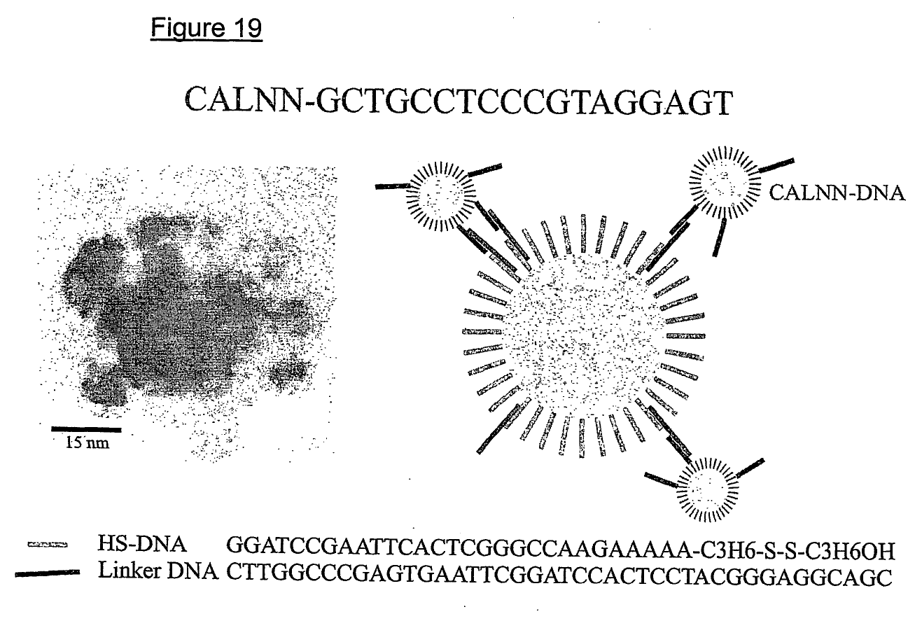

FIG. 19 shows an example of CALNN-DNA (hereafter peptide-DNA) as the functionalised peptide and CALNN (hereafter the matrix peptide as illustrated in Example 12;

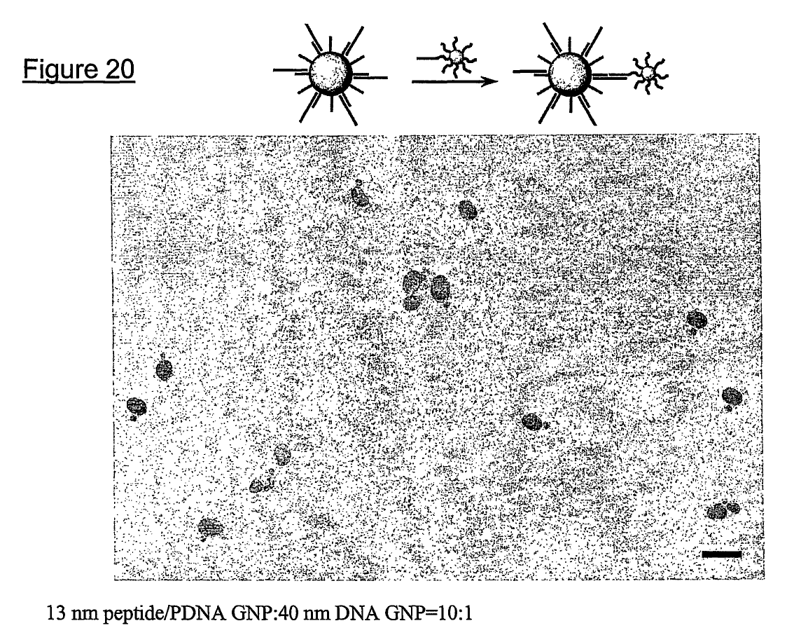

FIG. 20 shows an image of a simple assembly of nanoparticle conjugates as illustrated in Example 12;

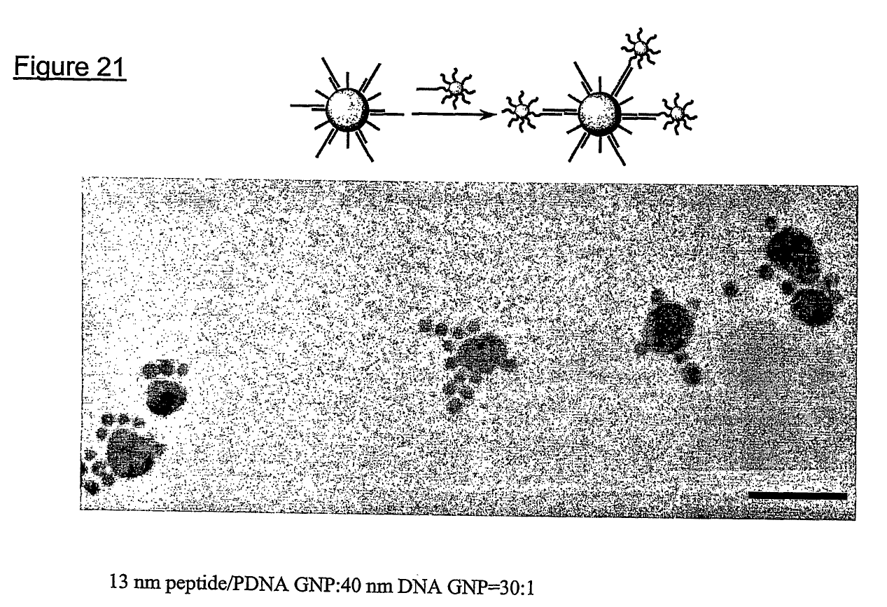

FIG. 21 shows an image of the effect of increasing the ratio of 13 nm peptide-DNA nanoparticles as illustrated in Example 12;

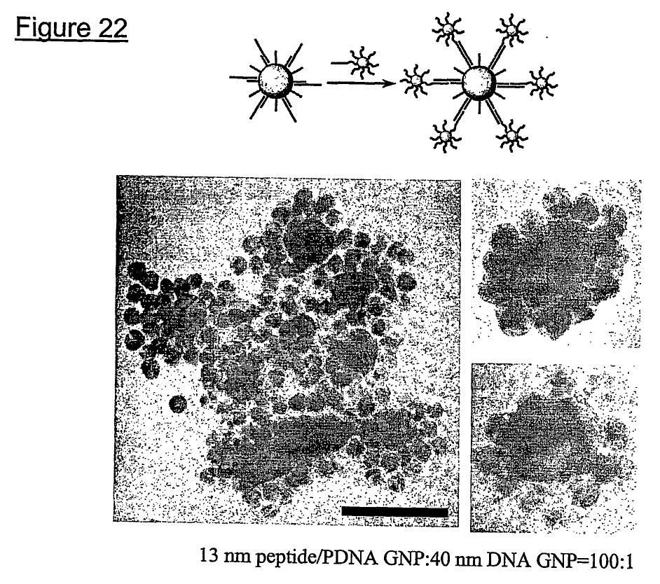

FIG. 22 shows an image of the effect of increasing the ratio of 13 nm peptide-DNA nanoparticles further than that shown in FIG. 21;

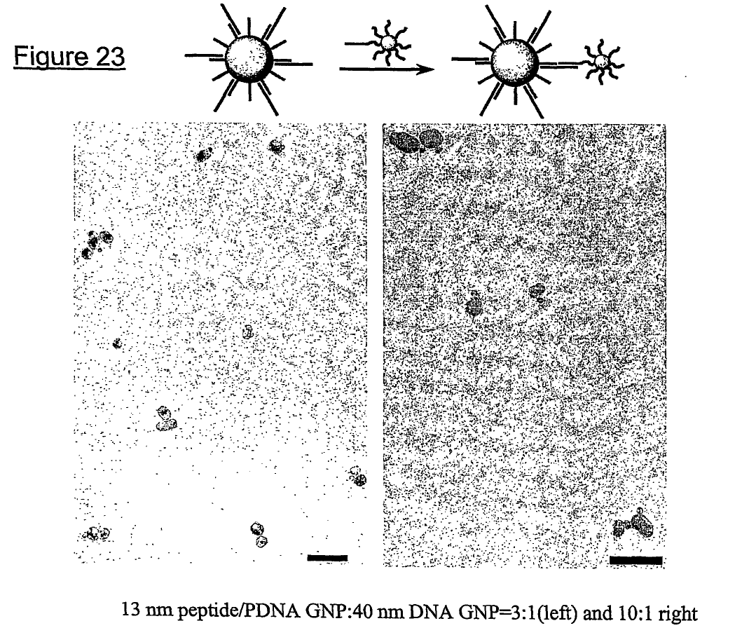

FIG. 23 shows an image of the effect of increasing the ratio of 13 nm peptide-DNA nanoparticles further than that shown in FIG. 22;

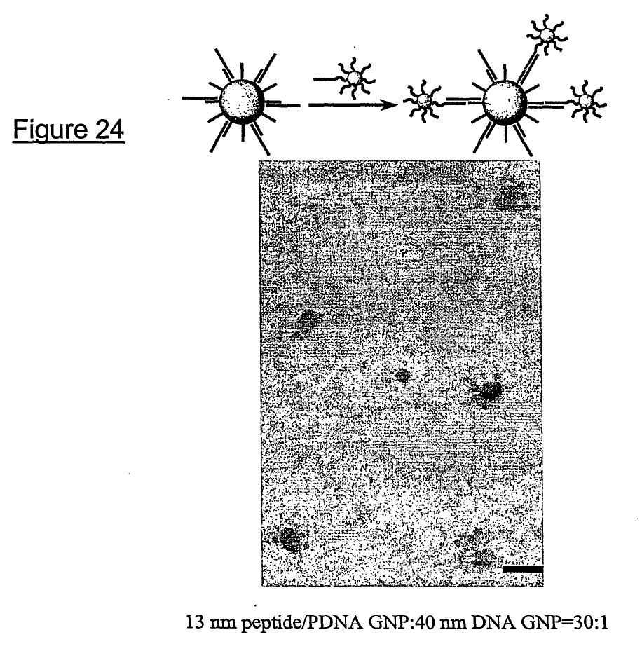

FIG. 24 shows an image of the effect of increasing the ratio of 13 nm peptide-DNA nanoparticles further than that shown in FIG. 23;

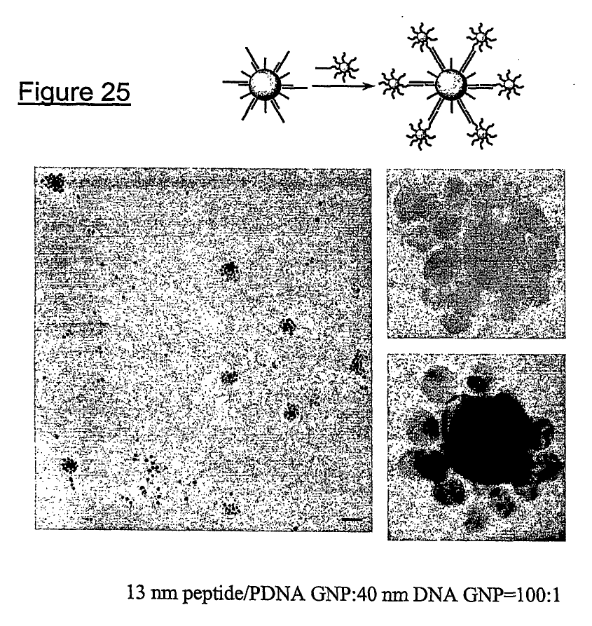

FIG. 25 shows an image of the effect of increasing the ratio of 13 nm peptide-DNA nanoparticles further than that shown in FIG. 24;

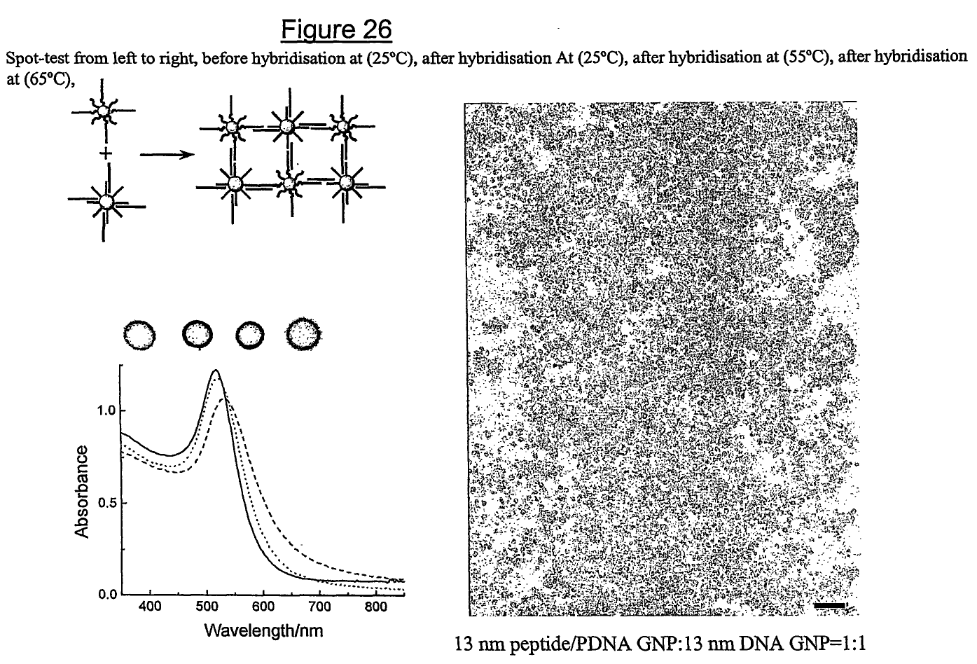

FIG. 26 shows an image of Peptide-DNA at 10.0% and ratio of nanoparticles 13 nm peptide/peptide-DNA:40 nm DNA=1:1 as illustrated in Example 12;

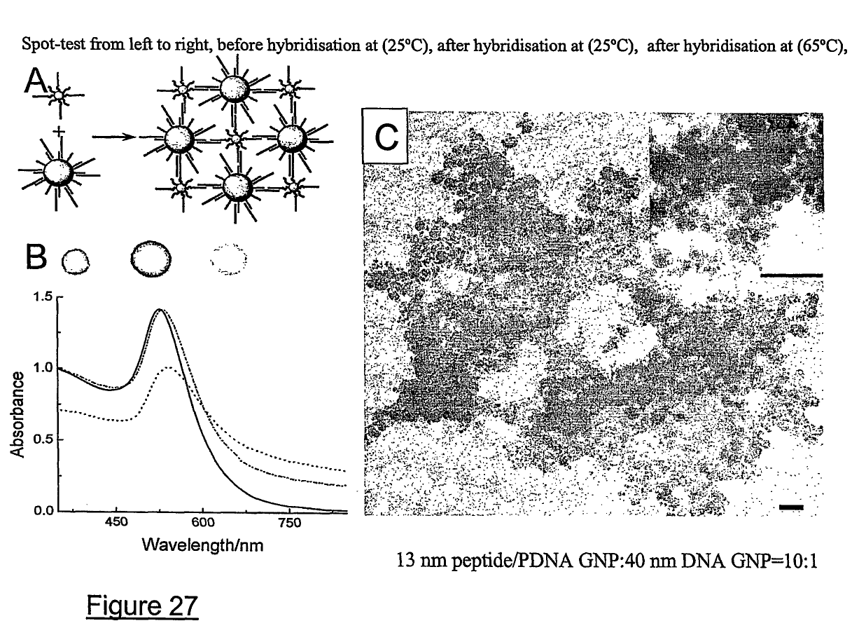

FIG. 27 shows an image of Peptide-DNA at 10.0% and ratio of nanoparticles 13 nm peptide/peptide-DNA:40 nm DNA=10:1 as illustrated in Example 12;

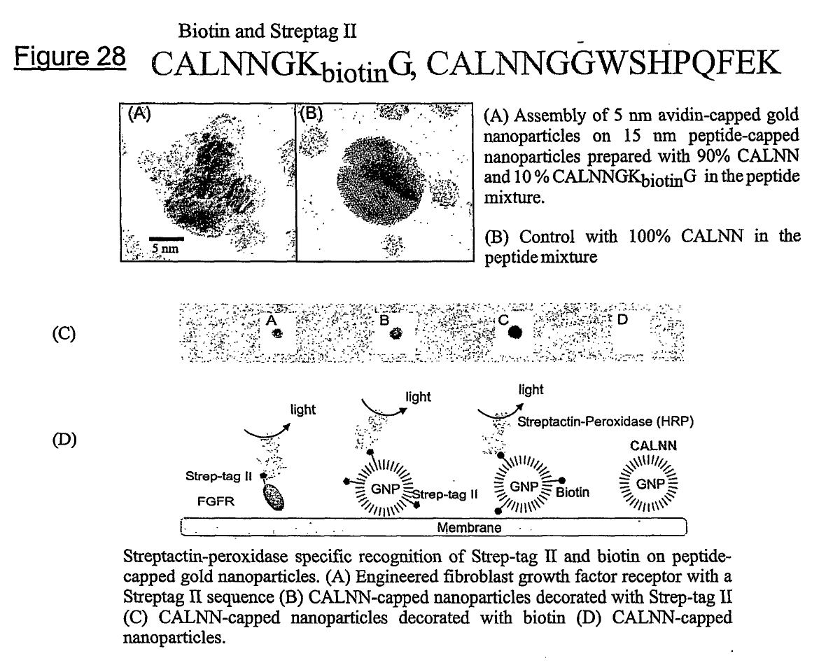

FIG. 28 shows the use of biotin and StrepTagII as functionalised extensions as illustrated in Example 12;

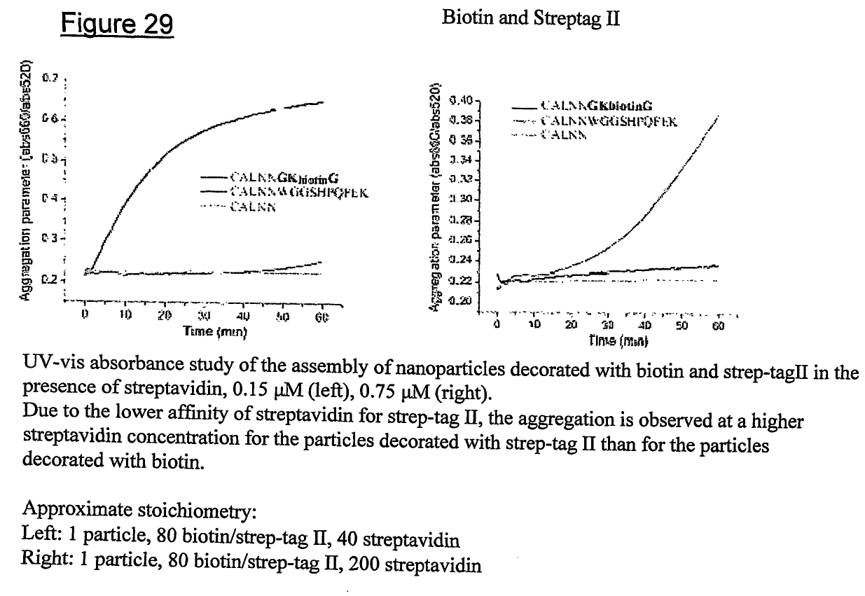

FIG. 29 shows the aggregation of 13 nm nanoparticles functionalised with biotin or StrepTagII by streptavidin determined by uv-vis absorbance as illustrated in Example 12; and

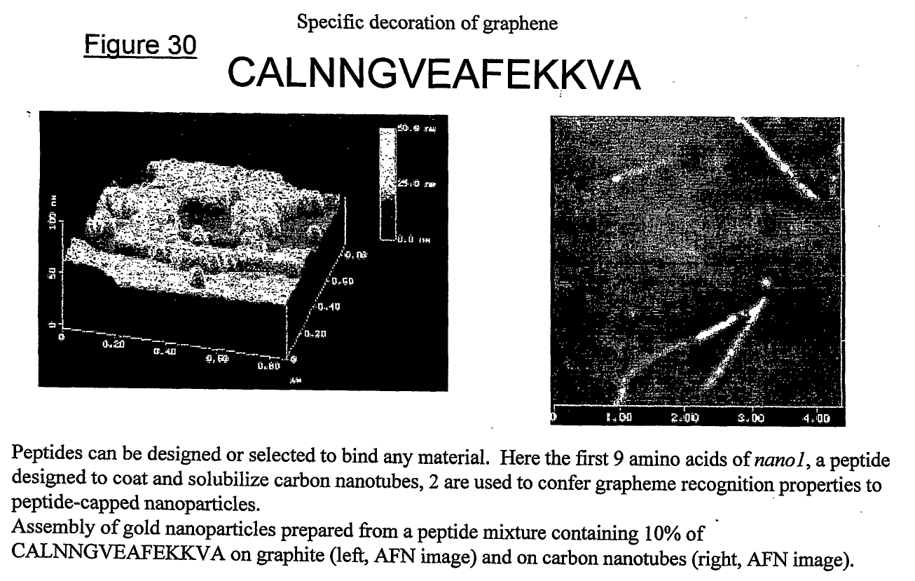

FIG. 30 shows the peptide extension that has been selected to recognise an artificial substance as illustrated in Example 12.

### EXAMPLE 1

An experiment was conducted to investigate the spectra of citrate and peptide capped 12.3 nm gold nanoparticles.

Citrate nanoparticles in water were mixed with 1 volume of phosphate-buffered saline with and without CALNN peptide. Spectra of 1 mL samples were analysed on a scanning spectrophotomer. The spectral shift caused by the adsorption of peptide to gold nanoparticles (in 50% PBS) was compared to citrate nanoparticles in water.

As shown in FIG. 1, the citrate nanoparticles were unstable in 50% PBS (70 mM NaCl, 5 mM Na₂HPO₄, pH 7.2), as evidenced by the appearance of an absorption band at 622 nm, characteristic of aggregated gold nanoparticles. In contrast, the presence of peptide in 50% PBS prevented the aggregation of gold nanoparticles. The mechanism appears to be due to the absorption of the peptide to the gold nanoparticle, since there is a 2.1 nm shift in the plasmon band, from 518 nm to 521.1 nm. Therefore the adsorption of peptide to the nanoparticle is surprisingly rapid compared to the adsorption of other thiolated ligands to gold. This unique virtually instant stabilisation of nanoparticles also appears to occur over a wide range of peptide concentrations.

### EXAMPLE 2

An experiment was conducted to perform size-exclusion chromatography of peptide capped 12.3 nm gold nanoparticles on a Sephadex G25 column.

As shown in FIG. 2, the nanoparticles stabilised with peptide were found to be separated from free peptide and citrate by size-exclusion chromatography on a Sephadex G25 size-exclusion column (chromatographic range 1000 Da to 5000 Da, void volume>5000 Da). The nanoparticles were eluted in the void volume, whereas the free peptide chromatographs near Vt and citrate eluted in a later peak just before Vt. As expected, if the concentration of nanoparticle in the coupling reaction is reduced while that of peptide remains constant, the nanoparticle and citrate peaks are reduced, while there is a small, but significant increase in free peptide peak, since less is adsorbed to the nanoparticles.

The peptide-capped nanoparticles purified by chromatography on G25 were free of excess peptide and citrate and are used in all the following experiments except that described in Example 4 and FIG. 4.

### EXAMPLE 3

An experiment was conducted to assess the stability of peptide capped nanoparticles over a range of pH and NaCl concentrations.

FIG. 3A shows the influence of pH on the stability of peptide capped 12.3 nm gold nanoparticles. The pH of solutions of peptide capped nanoparticles in 10% (v/v) PBS was adjusted and the spectra recorded after 5 min of incubation. The ratio of the absorbance at 522 nm (stable, single nanoparticles) and 622 (aggregates of nanoparticles) is used as a measure of nanoparticle stability. The peptide capped nanoparticles showed remarkable resistance to pH-mediated aggregation, since they are stable from pH 4 to pH 12.

FIG. 3B shows the influence of NaCl concentration on the stability of the nanoparticles. Nanoparticles were incubated for 5 min in different concentrations of NaCl, pH 7.0. Whereas there is no discernable spectral shift at 1 M NaCl, from 1.5 M the stability of the nanoparticles begins to be compromised, evidenced by the increase in absorbance at 622 nm. In 1 M NaCl, peptide capped nanoparticles were found to be stable for weeks.

### EXAMPLE 4

An experiment was conducted to assess the quantity by titration of the peptide adsorbed onto peptide capped 12.3 nm gold nanoparticles.

A titration to establish the total amount of absorbed peptide was conducted and then nanoparticles were removed by centrifugation. The concentration of free peptide in the supernatant was determined by measuring the absorbance at 190 nm of the supernatant. and this value was used to quantify the relative amounts of free peptide and therefore determine the approximate amount of bound peptide to the nanoparticle. FIG. 4 (I think this ffigure should be deleted as it cannot show this—call me Friday am at office (7954471) or home (7344014) if I am in late) shows that there are approximately 855±70 peptides per 12.3 nm diameter particle, although it is believed that with improvements to the protocol, up to 1500 peptides may be conjugated to a 12.3 nm diameter nanoparticle.

### EXAMPLE 5

An orientation experiment of the peptide on peptide capped 12.3 nm gold nanoparticles was conducted using anion-exchange chromatography on a column of DEAE-Sepharose.

Peptide capped nanoparticles were loaded onto a DEAE Sepharose fast flow column (0.5 ml) in 0.1 M NaCl (pH 7.2). The column was then eluted in steps of increasing NaCl (pH 7.2).

FIG. 5 shows that the nanoparticles were eluted at 0.4 M NaCl, which demonstrates that they are highly anionic. The only anionic group on the peptide is the C-terminal carboxylic acid. Normally such groups elute at far lower concentrations of NaCl, unless they are at high local concentration to produce avidity effects. Therefore, the majority, if not all of the peptides adsorbed to the nanoparticles were found to be oriented N-terminus on the gold surface and C-terminus exposed to solvent.

### EXAMPLE 6

An experiment was conducted to assess the specific association of nanoparticles driven by ligands on the capping peptide.

Nanoparticles were capped with a 10:1 ratio of standard peptide (CALNN) and standard peptide containing a three amino acid C-terminal extension and biotin, the structure of the nanoparticle conjugates comprising peptides with an extension to the core being CALNNK(biotin)GG. Following purification on Sephadex G25, the biotinylated 12.3 nm nanoparticles were mixed with 5 nm nanoparticles coated with avidin, these latter produced by standard methods, transferred to a TEM grid and images taken.

FIGS. 6A and 6B show a high relative ratio of 12.3 nm biotinylated nanoparticles and 5 nm avidin-coated nanoparticles. At low magnification (100 nm scale bar, panel A), the image shows that large complexes of nanoparticles are apparent. At higher magnification (10 nm scale bar, panel B) of a complex, it is clear that it consists of a large number of 12.3 nm nanoparticles. The bridging 5 nm avidin-coated nanoparticles are largely obscured.

FIGS. 6C and 6D show the results from the control experiment where the ratio of 12.3 nm and 5 nm nanoparticles is the same as in FIGS. 6A and 6B but the 12.3 nm nanoparticles are capped only with standard, non biotinylated peptide. In the absence of biotin on the 12.3 nm nanoparticles, no complexes between the two sizes of nanoparticles are apparent at either magnification and clearly demonstrating that the association of the 12.3 nm nanoparticles and 5 nm avidin coated nanoparticles is specifically driven by the biotin.

FIGS. 6E and 6F show a five-fold lower ratio of 12.3 nm nanoparticle to 5 nm avidin-coated nanoparticle in the mixture than in FIG. 6A-D. In FIG. 6(E) biotinylated 12.3 nm nanoparticles had been mixed with 5 nm avidin-coated nanoparticles and clearly formed complexes consisting of a core 12.3 nm nanoparticle surrounded by a few 5 nm avidin-coated nanoparticles. In FIG. 6F standard, non biotinylated peptide capped 12.3 nm nanoparticles were mixed with 5 nm avidin-coated nanoparticles. No association between 12.3 nm and 5 nm avidin-coated nanoparticles was found, again showing that their association is driven by the biotin-avidin interaction.

### EXAMPLE 7

A nanoparticle conjugate as outlined in the previous examples could be used as a basis for a sensor for ligand-induced dimerisation. FIG. 7A illustrates FGFR1-GPI (extracellular domain of Fibroblast Growth Factor receptor 1—glycosyl-phosphatidylinositol anchor) anchored to a HBM (hybrid bilayer membrane) and conjugated via a specific tag at the N-terminus to a nanoparticle. FIG. 7B shows induction of dimerisation of the receptor (FGFR-1) by the ligand (FGF-2 (Fibroblast Growth Factor), triangle) and Figure shows 7C the heparan sulfate (HS) co-receptor (chain). The use of nanoparticle-oligosaccharide conjugates (auroglycan) would provide a means to probe the function of the HS in the complex shown in FIG. 7D. The proximity of the nanoparticles brought together by the molecular interactions will result in their dipoles coupling and therefore in a redshift of the observed plasmon band and in the frequency of the light scattered by the nanoparticles. In principle the sensitivity is 1 nanoparticle or group of nanoparticles.

### EXAMPLE 8

Two experiments were conducted in order to produce nanoparticle conjugates wherein the peptide is incorporated during synthesis of silver nanoparticles, the two experiments comprising a two phase synthesis and a single phase synthesis.

A two phase synthesis with a phase transfer catalyst was performed by adding 9 mL of a 25 mM aqueous solution of silver nitrate to 7 mL of a 0.2 M solution of tetraoctylammonium bromide in toluene. This mixture was then stirred vigorously for 1 hour at room temperature. 1 mL of a 2 mg/mL solution of peptide in DMF (or DMSO, water, etc.) was then added to the mixture and stirred for 15 minutes. 7.5 mL of a 0.4 M aqueous solution of sodium borohydride was then added drop-wise and the solution was stirred for 2 hours. The phases were then separated and the aqueous phase filtered to remove Ag particles without the conjugated peptides.

A one phase synthesis was performed by adding 1 mL of a 2 mg/mL solution of peptide in water to 9 mL of a 25 mM aqueous solution of silver nitrate and the mixture was then stirred for 15 minutes. To the mixture, 7.5 mL of a 0.4 M aqueous solution of sodium borohydride was added drop-wise and the solution left to stir for 2 hours. The water was then filtered to remove Ag particles without the conjugated peptides.

In summary, in the two phase synthesis, the peptide is included in the aqueous phase, whereas in one phase synthesis the peptide is included in the reaction mixture. Nanoparticle conjugates appear in the aqueous phase after the reduction of the metal salt.

### EXAMPLE 9

A study was conducted to assess the protein folding considerations of the pentapeptide CALNN and variations thereof/related sequences, when conjugated to gold nanoparticles. The effect of 58 different peptide sequences conjugated to gold nanoparticles, the majority containing at least one cysteine residue for conjugation to the nanoparticle. The stabilities conferred by these peptides were found to be dependent on their length, hydrophobicity, and charge and in some cases resulted in further improved stability compared with CALNN.

The design of the peptide sequences took into account the need to have a strong affinity for godl, ability to self-assemble into a dense layer that substantially excludes water, and a hydrophilic terminus, which would ensure solubility and stability in water. As described in previous examples, the pentapeptide CALNN was initially developed and this pentapeptide was successful in meeting the requirements. The thiol group in the side chain of the N-terminal cysteine has the ability to make a covalent bond to the gold surface. Such an interaction may be additive to that of the N-terminal primary amine, since the amino groups are also known to have a strong interaction with gold surfaces. As it can be understood, CALNN is one of 3,200,000 possible sequences of five natural amino acids. Systematic variations of the peptide sequence were synthesised to assess the stability of the gold nanoparticles imparted by the different peptides. Nine sequences with no direct relation to CALNN were also tested, and these were based upon hydrogen bonding and P-strand formation and the presence of the thiol (cysteine) in the middle or at both ends of the peptides.

The following table lists the peptides that were synthesised and analysed:

TABLE 1

| | Anchor | | | | |
|---|---|---|---|---|---|
| | substitution | Core | Core | | |
| | in position | hydrophobic | hydrophilic | | |
| | 1, and | residues | residues | Terminal | |
| | reverse | substituted | substituted | substitution | |
| | sequence | in | in | in | |
| | of | positions | positions | positions | New |
| Length | CALNN | 2 and 3 | 2 and 3 | 4 and 5 | Design |
| CA | KALNN | CILNN | CKLNN | CALLS | CTTTT |
| CAL | AALNN | CLLNN | CDLNN | CALLD | CHRIS |
| CALN | CALNN | CVLNN | CTLNN | CALLK | CVVIT |
| CALNN | CCALNN | CFLNN | CNLNN | CALLR | CCVVVT |
| | NNLAC | CAANN | CAKNN | CALNS | CCVVVK |
| | | CAINN | CADNN | CALND | CALPDGLAAC |
| | | CAVNN | CATNN | CALNK | CVVITPDGTIVVC |
| | | CAFNN | CANNN | CALNR | NNLACALNN |
| | | CLANN | CDDNN | CALSS | NNLACCALNN |
| | | | CKKNN | CALSD | |
| | | | CTTNN | CALSK | |
| | | | CTSNN | CALSR | |
| | | | | CALKS | |
| | | | | CALKD | |
| | | | | CALKK | |
| | | | | CALLS | |
| | | | | CALSS | |
| | | | | CALKK | |
| | | | | CALNN | |

The effect of these 58 peptides on the electrolyte-induced aggregation of gold nanoparticles was studied. Since some of the peptides were very hydrophobic, dimethyl sulfoxide (DMSO) was used as the common solvent for their incorporation in the aqueous gold colloid solution, to facilitate the comparison of the sequences. After 1 hour, the pH was adjusted to 7 and the NaCl concentration progressively increased while the absorbance spectra were monitored.

For 19 of the investigated sequences, the addition of peptide induced an immediate aggregation of the particles. These peptides share the ability of bridging two particles together as a common structural feature. Most of them bear an amino (on the lysine, K) or guanidino (on the arginine, R) group distal to the thiol. These groups have a strong affinity for gold. NNLAC, the reverse sequence of CALNN, induces aggregation for the same reason. Apart from directionality, the only difference between these two peptides is that in CALNN the amino terminal group is on the cysteine bearing the thiol group, but in NNLAC the cysteine's thiol is adjacent to the terminal carboxylic acid and the amino terminal group is on the asparagine (N) in position 1. CAALPDGLAAC and CVVITPDGTIVVC were also found to induce aggregation.

During the study, the influence of the first amino acid (Anchorage) and peptide length was also assessed, and it was found that for a two amino acid peptide (CA), the aggregation parameter increases rapidly with NaCl concentration. As the length of the peptide increases from CA to CAL, CALN, and finally to CALNN, the NaCl-induced aggregation is displaced to increasingly higher concentrations of NaCl, suggestive of a direct correlation between peptide length and stability of the peptide-capped nanoparticles. Clearly, the thiol group plays a major role in stabilization, since the thiol-containing peptides CALNN and CCALNN show a much greater stability than KALNN and AALNN. Interestingly, aggregation of AALNN-capped nanoparticles occurs at higher concentrations of NaCl than for KALNN-capped nanoparticles. The higher density of terminal amino groups in KALNN may result in a degree of electrostatic repulsion between the peptides, which might prevent the formation of a self-assembled monolayer. Furthermore, hydrophobic interactions due to the additional methyl side chain of alanine in AALNN may also result in an increased stability of AALNN.

The influence of amino acids in Positions 2 and 3 was also assessed and the hydrophobic core (second and third amino acids) of the pentapeptide were changed into other hydrophobic amino acids (see Table 1, core, hydrophobic). The nine resulting peptides provided a degree of stabilization of the nanoparticles against NaCl-induced aggregation similar to that of the parental CALNN peptide). Even bulky aromatic amino acids such as phenylalanine (F) do not seem to disrupt the peptide self-assembled monolayer. Somewhat surprisingly, given that CALNN was designed with a bulkier hydrophobic group at position 3 to take into account particle curvature, bulky hydrophobic side chains (I and F) in position 2 appear to provide better stability with respect to NaCl-induced aggregation than when present in position 3. Thus, CILNN is more stable than CAINN and CFLNN is more stable than CAFNN. Charged (K and D) and neutral hydrophilic (T, S, and N) amino acids were substituted into the previously hydrophobic core (Table 1, core, hydrophilic). The presence of charged amino acids results in peptide sequences that generally provide poor protection against aggregation. For example, CDDNN-, CKLNN-, and CDLNN-capped nanoparticles aggregate at low NaCl concentration, although the presence of a negative charge in the third position (CADNN) seems to provide better stability than at the second position. In contrast, nanoparticles capped with peptides substituted with neutral hydrophilic amino acids, CNLNN, CANNN, CTLNN, CATNN, and CTTNN, generally aggregate at NaCl concentrations comparable to those with peptides possessing hydrophobic cores, although some combinations of neutral hydrophilic amino acids (CTSNN) were found not to be tolerated. In summary, the amino acids in positions 2 and 3 appear to require some attractive interaction between neighbouring peptide chains to provide stability. Thus, polar and hydrophobic amino acids promote the formation of a self-assembled monolayer through hydrogen bonding and hydrophobic interactions, respectively. However, the electrostatic repulsion of charged amino acids side chains in the core may prevent the formation of a dense peptide layer, hence leading to poor stabilization.

The influence of amino acids in positions 4 and 5 were also assessed and the role of the carboxylic acid terminus of the peptide (Table 1, end) was also addressed. Most of the sequences that were chosen for this study induce immediate aggregation of the nanoparticles upon addition of the peptide. Seven pentapeptide sequences provide a degree of stabilization Although there was found to be no obvious pattern between stability and the identity of the terminal two amino acids, these results indicated that the charge of the terminal amino acid contributes significantly to the stability of the peptide-capped nanoparticles. Introducing a second terminal negative charge (CALND, CALLD, CALSD, CALKD) reduces the concentration of NaCl that induces aggregation of the peptide-capped nanoparticles. The presence of the extra negative charge, and the absence of H-bonding between terminal amino acids, may affect the peptide packing leading to particles with a surface charge density similar to that of CALNN-capped nanoparticles, but with a less compact protecting peptide layer. H-bonding between the terminal amino acids is likely to play an important role, since replacing the penultimate N with a residue carrying a side chain that is a less amenable to H-bonding, e.g., CALND versus CALSD, reduces the stability.

The combinatorial analysis corroborates the initial design criteria. It establishes the need for a thiol (cysteine) as an anchor to the gold nanoparticle, a clear correlation between peptide length and stability, and the need for cohesive interaction between adjacent peptide chains through hydrophobic interactions or hydrogen bonding to provide high stability. The balance between peptide charge and cohesive interaction is shown to play a major role. The combinatorial analysis also allowed the definition of criteria for peptides leading to the immediate aggregation of the gold nanoparticles.

The other sequences, unrelated to CALNN which were also tested (CCVVVT, CVVIT, and CTTTT) were chosen for their strong β-strand forming properties. NNLACALNN and NNLACCALNN possess respectively one and two cysteines in the middle of their sequence. These two peptides should have an overall neutral surface exposed with the carboxylic and amino terminal groups at the peptide-water interface, since it would be expected that bonding will preferentially occur at the central thiol in the cysteine. The presence of the second cysteine (C) greatly improves the stability, maybe by imposing a peptide configuration more favourable to packing. Two of the β-strand forming peptides, CCVVVT and CTTTT, also show very promising behaviour, with small values of the aggregation parameter at 500 mM NaCl. In particular, CCVVVT has a greater stability than CALNN with no indication of aggregation at 500 mM NaCl. Considering the relatively small number of sequences tested, the use of larger libraries will allow the identification of capping ligands with even better properties.

### EXAMPLE 10

An experiment was conducted to prepare and characterise water soluble magnetic nanoparticles capped with a number of peptides for bio-applications. In particular, a range of cobalt materials were analysed along side other magnetic materials.

Cobalt nanoparticles have been synthesized with full control of size (Puntes, V. F., et al., (2001) Science 291: 2115-2117), and have the desired high magnetic moment and susceptibility. However, they are only stable in organic solvent environments; in water they oxidize to give Co²⁺. These species (such as cobalt hydroxide), do not possess the magnetic properties of the metal. The two main approaches for making magnetic particles soluble in water are in situ synthesis and phase transfer. In situ synthesis of Co nanoparticles in the presence of peptides is described as a route to producing water soluble Co nanoparticles, in addition to an obvious exchange reaction, analogous to that employed the Au-citrate nanoparticles. The rapid self-assembly of peptides into a close packed ligand shell has been shown to stably protect other metallic nanoparticles (Levy, R., et al., (2004) J. Am. Chem. Soc., 126, 10076-10084). Therefore, introduction of recognition groups on the surface of nanoparticles may be easily achieved by including a defined percent of a modified peptide possessing a specific tag during the ligand-exchange/synthesis. There are several possible protocols: one is the Puntes paper, but replacing the oleic acid/TOPO ligand in the organic solvent with peptide dissolved in DMF or DMSO—this gives the material in the TEM. The other method is similar to the Au, but differs in detail since the Au are citrate stabilised and in water, whereas the Co start off in solvents described in the Puntes paper, e.g, dichlorobenzene, toluene. For example:
1. Wash Co nanoparticles with solvent to remove all excess ligand. In practice this is oleic acid and TOPO as described in the Puntes paper, but could be anything else developed in the future.
2. Add peptide dissolved in DMASO or DMF
3. Mix and leave to allow ligand exchange. This is relatively show compared to Au or Ag
4. remove solvent
5. Add water.
Nb: washing of Co nanoparticles can make use of their magnetism: a magnet can be placed next to the tube, and the liquid phase removed.

The following table lists the magnetic properties of different materials and illustrates that Co nanoparticles combine a small size with high magnetisation saturation and so are a much sought after material:

TABLE 2

| | | Ms at | Standard | Synthesis | Critical size to be | |
|---|---|---|---|---|---|---|
| | | 300 K | Red. Pot. | routes | superparamagnetic | |
| | Materials | (Gauss) | E0/V | For NP | at Room temp (nm) | Over all |
| 3d | α-Fe | 1707 | Fe2+/Fe: −0.44 | Yes | 17 | No |
| transition | Co | 1400 | Co2+/Co: −0.28 | Yes | 8.1-11.6 | Yes |
| metals | Ni | 485 | Ni2+/Ni: −0.257 | Yes | 34 | No |
| | | | Au+/Au: +1.83 | | | |
| Metallic | Fe23B6 | 1350 | | No | 28.7 | No |
| Alloys and | Fe3B | 1280 | | No | 10.6 | No |
| compounds | CoPt | 800 | | Yes | 3.6 | No |
| | FePt | 1140 | | Yes | 2.4-3.2 | No |
| Oxides | Fe3O4— | 480 | | Yes | 27.8 | No |
| | magnetite | | | | | |
| | γFe2O3— | 420 | | Yes | 36.2 | No |
| | Maghemite | | | | | |
| | CoO | — | | No | — | No |
| | Co3O4 | — | | No | — | No |

The following peptides were conjugated with the nanoparticles in situ during nanoparticle synthesis to test the suitablility of the cobalt nanoparticles for use in accordance with the present invention:

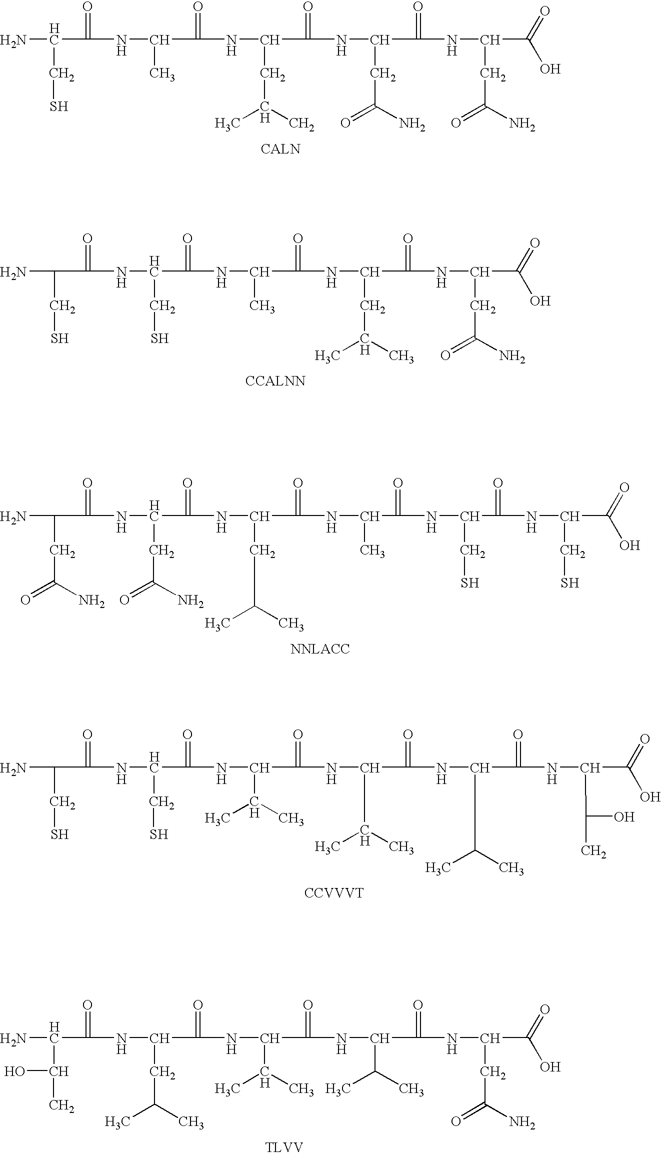

The samples were examined by bright-field (BF) and dark-field (DF) transmission electron microscopy and the nanoparticles can be seen in FIG. 8. The dark field view of FIG. 8 consists of observing the image produced by the diffracted electrons instead of the transmitted ones. Dark-field imaging provides direct observation of the metallic nanoparticles (NP) within the organic matter. It was found that different types of compounds deposited onto the substrate: metallic particles of 7 nm embedded in an organic (peptide) droplet, large aggregates and individual nanoparticles, and combinations of these. Self-assembly is not observed in contrast to cases where hydrophobic NP are evaporated from organic solvents onto hydrophobic substrates.

FIG. 9 shows X-ray diffraction patterns (Co ka=1.798) for different synthesises where hcp and epsilon Co crystal structures appear, pure or mixed with Co oxides and hydroxides (* peaks). The broadness of the peaks is related to their nanometric size.

The nanoparticles were analysed by SQUID [SQUID=instrument not a method] and the ZFC/FC measurements (FIG. 10) show a peak in the ZFC curve at around 6 K. This is evidence for NP less than a few nanometers in diameter in the sample. The moments of these small particles become blocked at low temperatures and so lead to the observed ZFC/FC curves. The samples, however, also contain larger sized NP with a wide size distribution. As a consequence the ZFC and FC curves are split not at the position of the peak (6K), but at much high temperature. Even at room temperature we observed an open hysteresis loop (FIG. 10 insert), which indicates that some particles of around 10 nm diameter are also present in the sample.

In exchange experiments, 10 nm Co nanoparticles synthesized with oleic acid and TOPO (Puntes Science ref cited earlier) were washed in toluene and resuspended in this solvent and then mixed with peptide. All 58 peptides from the library were tested. The state of aggregation of the nanoparticles was followed over 6 days. After this they were freeze-dried and resuspended in water. Some peptides, of which CALNN was the most effective enabled the nanoparticles to be resuspended in water with retention of the magnetic properties. As an alternative to freeze-drying, the solvent can simply be removed from the Co nanoparticles and replaced with water.

### EXAMPLE 11

An experiment was conducted to prepare and characterise silver (Ag) nanoparticles capped with a number of peptides by the exchange of peptide ligands onto citrate-Ag nanoparticles.

As with Au particles, the stabilisation of the Ag nanoparticles is instantaneous and produces extremely stable nanoparticles. Given the propensity of Ag to oxidise in aqueous environments, the long-term stabilisation of Ag nanoparticles by peptides provides an even more stringent test of the remarkable properties of the peptide ligands. Stability is measured 10-15 minutes (hereafter ‘short-term”) and 24 h (hereafter “long-term”) after the addition of peptide. In practice, preparations of particles showing long-term stability are stable for months. Standard nanoparticle preparations are in 10 mM Tris, pH 8.0, unless stated otherwise; NaCl is additional to this buffer. The results of the experiments can be seen with reference to following figures:

FIG. 11 Ag-CALNN: The pentapeptide CALNN was added to an aqueous solution of citrate-Ag nanoparticles. The citrate Ag nanoparticles were 15.3±6.4 nm in diameter. Solutions of the purified particles were tested at different pH values (A) and concentrations of NaCl (B).[(Simon, control showed this was buffer]. Panel A shows that the particles exhibit long-term stability between pH 4 and 12. Panel B shows that the particles are stable at equal to or less than 1 M NaCl. This is true for both the short- and long-term.

FIG. 12 Ag-CCALNN: The hexapeptide CCALNN was added to an aqueous solution of citrate-Ag nanoparticles. Solutions of the purified particles were tested at different pH values (A) and NaCl concentrations (B). Panel A shows that the particles are stable between pH 4 and 12. This is true for both the short- and long-term. Panel B shows that the particles are stable up to and including 1 M NaCl. This is true for both the short- and long-term.

FIG. 13 Ag-CVVVT: The pentapeptide CVVVT was added to an aqueous solution of citrate-Ag nanoparticles. Solutions of the purified particles were tested at different pH values (A) and salt concentrations (B). Panel (A) shows that the particles exhibit short-term stability between pH 2 and 12, with a shift of the plasmon peak for pH 2 through 6. The particles do not display long-term stability at pH 2 and 3. Panel B shows that the particles are stable up to and including 1M NaCl. This is true for both the short- and long-term.

FIG. 14 Ag-CCVVVT: The hexapeptide CCVVVT was added to an aqueous solution of citrate-stabilized Ag nanoparticles. Solutions of the purified particles were tested at different pH values (A) and NaCl concentrations (B). Panel A shows that the particles exhibit short-term stability between pH 2 and 12, with a shift of the plasmon peak for pH 2 through 6. The particles do not display long-term stability at pH 2 and 3. The bottom graph shows that the particles are stable up to and including 1M NaCl. This is true for both the short- and long-term.

CALNN-capped Ag nanoparticles were produced in a two phase modified Brust synthesis. Briefly, silver is reduced in the presence of peptide in a two-phase mixture of toluene, water, and a phase-transfer catalyst.

FIG. 15 Ag-CALNN: Solutions of the purified particles were tested at different pH values (A) and NaCl concentrations (B). Panel A shows that the particles exhibit short-term stability between pH 3 and 12. Particles at pH 3 do not display long-term stability. Panel B shows that the particles are stable at 1M NaCl. This is true for both the short- and long-term.

FIG. 16 TEM of Ag particles: Panels A-C Ag nanoparticles capped with CALNN produced by exchange with citrate. TEM images of the CALNN-capped Ag nanoparticles produced by exchange with citrate. The majority of the particle diameters were found to be between 10 and 20 nm and have a size distribution of 16.3±4.5 nm in diameter (mean±SD). The particles are mostly spherical in shape and display good shape uniformity across the size range. Panels D-F Ag nanoparticles produced in two phase synthesis with CALNN. TEM images of the CALNN-capped Ag nanoparticles produced in a 2-phase synthesis and used in the previous stability studies. The majority of particle diameters are between 5 and 15 nm and have a size distribution of 8.2±2.4 nm in diameter (mean±SD). The particles are mostly spherical in shape and display good shape uniformity across the size range.

FIG. 17 Separation of Ag-PEG and Au-CCALNN on a CL-6B column: Since the synthesis of Ag nanoparticles does not result in relatively monodisperse particles, like the synthesis for Au nanoparticles, size separation on gravity-driven gel filtration columns is employed to reduce the size distribution. The experiment demonstrates a proof-of-concept experiment using Au and Ag nanoparticles, since these are readily detected as separate entities by eye. Twelve nm CALNN-capped Au nanoparticles (red) are separated from tetraethylene glycol-capped Ag nanoparticles with diameters between 3 and 7 nm (yellow). (A-C) Progress of chromatography on a column of CL-6B, showing the gradual separation of the larger Au nanoparticles from the smaller Ag nanoparticles. (D) One mL fractions collected from the CL6B column show clear separation of the two sizes of particles.

### EXAMPLE 12

An experiment was conducted to functionalise Au peptide-capped nanoparticles.

The peptide ligands have two unique properties. The first is their ability to stabilise nanoparticles instantaneously. The second is that they provide a unique route to functionalising nanoparticles in a controlled fashion by producing nanoparticles with a defined valency. These experiments provide some instances of peptide functionalisation and the peptide-DNA work demonstrates the principle of controlling the valency of functionalisation. The results of the experiments can be seen with reference to the following figures:

FIG. 18 shows a schematic representation of the principle of using a matrix peptide (CALNN in this case) for stabilisation and a defined mole percentage of one or more peptide species (CALNNXXX and CALNNYYY in the example given) carrying an extension that imparts a specific functionality (usually a recognition function). The number of peptide ligands per nanoparticle is known (855±70 for 12.3 nm nanoparticles) and this number is used to choose the ratio of matrix peptide:functionalised peptide. For example, a ratio of 1000:1 will produce a majority of nanoparticles with one functionalised peptide. Single valency substitution is achieved by using higher ratios, e.g., 3000:1, and chromatographic separation based on the unique properties of the extension (in practice affinity chromatography) to remove the non-functionalised nanoparticles. Using an elution gradient in affinity chromatography and a lower peptide ratio, nanoparticle species of different valencies may be purified (,di-, tri- etc.- valent).

FIG. 19 shows an example of CALNN-DNA (hereafter peptide-DNA) as the functionalised peptide and CALNN (hereafter the matrix peptide). HS DNA is standard thiolated DNA used to functionalise nanoparticles with DNA. The linker DNA is complementary to the single stranded HS DNA on the large nanoparticle and the peptide-DNA on the small nanoparticle.

What follows are examples of complexes formed between 40 nm nanoparticles with a HS-DNA ligand and 12.3 nm nanoparticles with matrix peptide and peptide-DNA. By simply controlling the ratio of 40 nm to 12.3 nm nanoparticles and the peptide-DNA as a percentage of matrix peptide it is possible to control the assembly of the nanoparticles. This route is the only one to combine such control of the state of assembly of the nanoparticles with incredible ease of preparation.

FIG. 20 shows Peptide-DNA at 0.3% and ratio of nanoparticles 12.3 nm peptide/peptide-DNA:40 nm DNA=10:1. At 0.3% peptide-DNA there will be ˜3 peptide-DNA per nanoparticle. Simple-assemblies are produced consisting of a single 40 nm HS-DNA nanoparticle to which are bound one or just a few 12.3 nm peptide-DNA nanoparticles.

FIG. 21 shows the effect of increasing the number of 12.3 nm peptide-DNA nanoparticles. The ratio of nanoparticles 12.3 nm peptide/peptide-DNA:40 nm DNA=30:1, peptide-DNA at same percentage as in FIG. 20, 0.3%. With more small nanoparticles, the assemblies are still simple, but consist of a central 40 nm HS-DNA nanoparticle almost completely surrounded by small 12.3 nm peptide-DNA nanoparticles.

FIG. 22 shows the effect of further increasing the number of 12.3 nm peptide-DNA nanoparticles. Ratio of nanoparticles 12.3 nm peptide/peptide-DNA:40 nm DNA=100:1, peptide-DNA at same percentage as in FIG. 21, 0.3%. With far more small nanoparticles, the assemblies are still simple, but now consist of a central 40 nm HS-DNA nanoparticle (Simon, this one has more than the one before!) completely surrounded by small 12.3 nm peptide-DNA nanoparticles.

FIG. 23 shows Peptide-DNA at 1.0% and ratio of nanoparticles 12.3 nm peptide/peptide-DNA:40 nm DNA=3:1 and 10:1. At 1.0% peptide-DNA there will be ˜9 peptide-DNA per nanoparticle. The higher valency of the 12.3 nm peptide-DNA nanoparticles is reflected by the fact that at particle ratios of 12.3 nm peptide DNA nanoparticle:40 nm HS-DNA nanoparticle of 3:1 and 10:1, the assemblies are more complex than seen in FIGS. 20-22, since bridging of one or more large HS-DNA 40 nm nanoparticles by 12.3 nm peptide-DNA nanoparticles is evident.

FIG. 24 shows the effect of increasing the number of 12.3 nm peptide-DNA nanoparticles. Ratio of nanoparticles 12.3 nm peptide/peptide-DNA:40 nm DNA=30:1, peptide-DNA at same percentage as in FIG. 23, 1.0%. As the concentration of 12.3 nm peptide nanoparticles increases, they almost surround the 40 nm HS-DNA particles and prevent bridging.

FIG. 25 shows the effect of increasing the number of 12.3 nm peptide-DNA nanoparticles. Ratio of nanoparticles 12.3 nm peptide/peptide-DNA:40 nm DNA=100:1, peptide-DNA at same percentage as in FIG. 24, 1.0%. As the concentration of 12.3 nm peptide nanoparticles increases, they now effectively completely (again I feel that effectively used in the previous para is stronger than almost, used in this para) surround the 40 nm HS-DNA particles and prevent bridging.

The following experiments produce large aggregates, which are of the type observed when solely HS-DNA nanoparticles are used. As expected, heating the aggregates to above the melting temperature reverses the DNA hybridisation driven aggregation.

FIG. 26 shows Peptide-DNA at 10.0% and ratio of nanoparticles 12.3 nm peptide/peptide-DNA:40 nm DNA=1:1. The peptide-DNA nanoparticles now have a valency of nearly 90, at which point they form large aggregates with the 40 nm HS-DNA nanoparticles, which have an apparent periodicity.

FIG. 27 shows Peptide-DNA at 10.0% and ratio of nanoparticles 12.3 nm peptide/peptide-DNA:40 nm DNA=10:1. Increasing the number of 12.3 nm peptide-DNA nanoparticles increases the apparent ‘density’ of the aggregates.

FIG. 28 illustrates the use of biotin and StrepTagII as functionalised extensions. Biotin binds avidin, streptavidin, streptactin, etc., whereas the StrepTagII sequence only binds streptavidin and streptactin. A, B, TEM of assembly of small 5 nm nanoparticles coated with streptavidin and 13 nm nanoparticles with 10% CALNNK(biotin)GG, where the biotin is on the ε amino group of the lysine residue's side chain and a control in which the 12.3 nm nanoparticles have only CALNN. C, D, recognition of StrepTagII on 12.3 nm nanoparticles by streptactin-peroxidase. Blot “D” shows that there is no detectable non-specific binding of streptactin peroxidase by 12.3 nm nanoparticles with only CALNN.

FIG. 29 shows aggregation of 12.3 nm nanoparticles functionalised with biotin or StrepTagII by streptavidin determined by uv-vis absorbance. The aggregation parameter is as defined in Lévy et al., (J. Am. Chem. Soc. 2004). The interaction of biotin with streptavidin is of higher affinity and proceeds with faster kinetics than that of streptagII. Therefore the ratio of nanoparticles:streptavidin that produces aggregates is different.

FIG. 30 shows an example of a peptide extension that has been selected to recognise an artificial substance. The sequence of the peptide “nano1” was identified as recognising specifically single walled carbon nanotubes. Nanoparticles (12.3 nm) with 10% CALNN-nanol bind specifically to single walled carbon nanotubes.

### EXAMPLE 13

An experiment was conducted to prepare thiol-derivatised protein-like gold nanoparticles, via the formation of mercury adducts, so as to generate versatile “glycoconjugates”. The experiments show an alternative approach to producing nanoparticle conjugates in accordance with the present invention, which relies on the high-yielding derivatisation of oligosaccharides obtained by enzymatic degradation of GAGs with lyases and the attack of the resulting unsaturated bond in the non-reducing end uronic acid by mercury salts and the subsequent attachment of the mercury adduct to a thiol group of a carrier molecule or surface to form stable glycoconjugates. The approach is illustrate by the preparation of heparan sulfate-gold nanoparticle conjugates.

The typical reaction for the preparation of Au-CALNN-Met involved adding 50 μL of EDC (1M) to 400 μL of Au-CALNN (OD˜0.32) while vortexing, and the reaction tube was left to stand for 15 min. Then 50 μL of Met (0.33 M) was added to the reaction mixture and left for 1 hr. Excess reagents were removed by dialysis in a Slide-A-Lyser dialysis cassette (Pierce) over night in 1 L of phosphate buffer.

In this example, for the preparation of Hg-Sugar, a model hexaccharide, termed “degree of polymerisation 6 (DP6: 0.25 mg, 0.63 μmoles) derived from heparan sulfate by heparitinase enzyme digestion and bearing an unsaturated 4,5 uronic acid at the non-reducing end was reacted in water/THF (1 mL, 1/1 (v/v) 2 hr, 40° C. with Hg(OAc)2 (1 mg, 3 μmol).

Preparation of Au-CALNN-Met-S . . . Hg-sugar was by adding 1 μL of DP6-Hg to 100 μL of Au-CALNN-Met the reaction was monitored after 30 min.

The results of the experiments are listed in the following table (Table 3):

TABLE 3
Experiments and controls for reaction of Au-CALNN-Met with DP6

| Reactions | Spectrum change |
|---|---|
| Au-CALNN-Met + sucrose (non reducing end) | NO |
| Au-CALNN-Met + sucrose + Hg (OAc)2 | NO |
| Au-CALNN-Met + Heparin | NO |
| Au-CALNN-Met + Heparin + Hg (OAc)2 | YES |
| Au-CALNN-Met + sugar (DP6) | NO |
| Au-CALNN-Met + Hg-sugar (DP6) | YES |

The results of the experiments can be summarized by the following points:
1. High yielding mercury adduct of HS saccharide made-structure confirmed by MALDI-MS (FIG. 6) {simon, this canot be FIG. 6??), ¹H and ¹³C NMR, which also show Hg attached (>95% pure).
2. Preparation of Au NP coated with CALNN and CALNNMet. Cys could also be used, but its thiol is more generally reactive than the thioether of met, although both react equally with Hg2+.
3. Evidence for the formation of AuNP-CALNN-Met-S . . . Hg-DP6 is shown in FIG. 7 (Simon, fig number???). Prior to coupling, the Hg-DP6 was purified twice in the presence of EDTA (100 mM) to chelate free Hg 2+ ions. Comparison of the spectra of AuNP-CALNN and AuNP-CALNNMetS . . . Hg-DP6 show a difference in λmax of 2 nm with a reduction in intensity and increased absorbance at longer wavelength characteristic of particle aggregation.
4. Control experiments of all other combinations of Au NP or AuNP-CALNN with either Hg ion, Sugar and Hg-Sugar failed to show any change in the U.V.-vis spectrum. Table 3 shows there is an interaction between Au-CALNN-Met and heparin in the presence of Hg(OAc)₂. This might due to the interaction of the Hg ion with the sulphate groups of heparin. Consequently, EDTA was used to remove free Hg ions from the Hg-DP6 conjugate. This confirms that the changes in optical characteristics are specific to the presence of Hg-DP6 and are consistent with its attachment to the peptide via Met on the Au surface.
5. From the results of these experiments the creation of analogues of heparan sulfate proteoglycans for a number of applications will be possible, such as the investigation of growth factor signalling, multivalent inhibitors of attachment of pathogenic microorganisms to host cell heparan sulfate and novel strategies for sequencing heparan sulfate oligosaccharides.

1. A nanoparticle conjugate comprising a nanoparticle conjugated to a plurality of peptides of a substantially similar amino acid sequence, wherein the peptides are conjugated to the nanoparticle by means of a Cysteine (C) residue and the nanoparticle conjugate further comprises ligands attached to at least one said peptide.

2. The nanoparticle conjugate of claim 1, wherein the Cysteine (C) residue is conjugated to the nanoparticle by means of its thiol and/or amino group.

3. The nanoparticle conjugate of claim 1, wherein the distal end of the peptide terminates with a carboxyl group or the peptide is conjugated to the ligand by means of a carboxyl group.

4. The nanoparticle conjugate of claim 1, wherein the peptide independent of the ligand has the sequence CALNN or CCALNN.

5. The nanoparticle conjugate of claim 1, wherein at least one peptide ligand moiety has the sequence
CXn(ligand),
CCXn(ligand),
CXn(ligand)Xn, or
CCXn(ligand)Xn
wherein X denotes any amino acid residue and n denotes any length of amino acid residues.

6. The nanoparticle conjugate of claim 1, wherein at least one Cysteine residue is located in a central region of the peptide.

7. The nanoparticle conjugate of claim 6, wherein there are two or more Cysteine residues located in a central region of the peptide.

8. The nanoparticle conjugate of claim 6, wherein in at least one peptide sequences on either side of the Cysteine residue are substantially symmetrical.

9. The nanoparticle conjugate of claim 6, wherein at least one peptide has the sequence
(ligand)XnCXn(ligand),
Xn(ligand)XnCXn(ligand)Xn,
(ligand)XnCCXn(ligand) or
Xn(ligand)XnCCXn(ligand)Xn
wherein X denotes any amino acid residue and n denotes any length of amino acid residues.

10. The nanoparticle conjugate of claim 9, wherein at least one peptide independent of the ligand has the sequence NNLACALNN or NNLACCALNN.

11. The nanoparticle conjugate of claim 1, wherein the nanoparticle conjugate further comprises an identification means attached to at least one peptide or the ligand.

12. The nanoparticle conjugate of claim 1, wherein the nanoparticle conjugate further comprises a functional group attached to at least one peptide or ligand.

13. All The nanoparticle conjugate of claim 11, wherein an additional sequence of amino acid residues is disposed between the ligand and the identification means and/or after the ligand or identification means.

14. The nanoparticle of claim 13, wherein the additional sequence comprises two or more Glycine residues.

15. The nanoparticle conjugate of claim 1, wherein the nanoparticle conjugate comprises different peptides.

16. The nanoparticle conjugate of claim 15, wherein different ligands and optionally different identification means and/or functional groups are attached to said different peptides.

17. The nanoparticle conjugate of claim 1, wherein the nanoparticle conjugate is capable of being conjugated to at least one other nanoparticle conjugate or is conjugated to at least one other nanoparticle conjugate to form nanoparticle conjugate assemblies.

18. The nanoparticle conjugate of claim 1, wherein the nanoparticle is produced from a metallic material, a magnetic material or a semi-conducting material.

19. The nanoparticle conjugate of claim 1, wherein nanoparticle is produced from a gold, silver, cobalt, nickel, platinum, cadmium selenide or zinc sulphide.

20. The nanoparticle conjugate of claim 1, which further contains an identification means and/or functional group and wherein said identification means and/or functional group and/or ligand is selected from one or more of: biotin and/or avidin, streptavidin, streptactin, a histidine tag, NTA, radioactive labels, antigens, epitopes or parts of epitopes, antibodies, fluorochromes, nucleic acids, recognition sequences, enzymes, antibodies, peptides, proteins, receptors or a target molecules, saccharides, polysaccharides and lipids.

21. The nanoparticle conjugate of claim 12, wherein an additional sequence of amino acid residues is disposed between the ligand and the functional group and/or after the ligand or functional group.

22. The nanoparticle conjugate of claim 1, wherein an additional compound or pharmaceutically active salt is conjugated to the nanoparticle or to at least one peptide.

23. The nanoparticle conjugate of claim 1, wherein the nanoparticle has a diameter in the range of 1-100 nm.

24. The nanoparticle conjugate of claim 1, wherein the plurality of peptides substantially cover the surface of the nanoparticle so as to provide a shell around the nanoparticle.

25. The nanoparticle conjugate of claim 24, wherein wherein the shell permits optical detection of the nanoparticle including coupling effects for multiple nanoparticles.

26. A nanoparticle conjugate of claim 1, which contains approximately 500-1500 peptides per nanoparticle.

27. The nanoparticle conjugate of claim 1, which contains approximately 1.0-3.2 peptides per nm².

28. A method to carry out a process selected from the group consisting of diagnostic assay, or separation and/or purification of proteins, or production of therapeutic agents,
which comprises employing the nanoparticle conjugate of claim 1 in said process.

29. A method to carry out a process selected from the group consisting of chromatography, ELISA, lyophilisation, FISH, ISH, SDS PAGE, flow cytometry, immunohistochemistry, Western blotting, cytogenetic analysis, molecular interaction assays, histochemistry on fixed and living cells/tissue or high throughput screening,
which comprises employing the nanoparticle conjugate of claim 1 in said process.

30. A method to produce a nanoparticle conjugate of claim 1, which comprises incubating in water, nanoparticles stabilised in a stabilising solution with a plurality of peptides in all phosphate buffered saline or other combination of solvent electrolytes.

31. The method of claim 30, wherein one or more ligands and optionally one or more identification means and/or functional groups are conjugated to the peptide prior to incubation with the nanoparticle or during the course of the incubation.

32. A method to produce a nanoparticle conjugate of claim 1, which comprises including a plurality of peptides comprising one or more ligands and optionally one or more identification means and/or functional groups during the synthesis of the nanoparticle.

33. The method of claim 32, wherein the nanoparticle conjugate is freeze dried for storage.

34. (canceled)

35. A nanoparticle conjugate comprising a nanoparticle conjugated to a plurality of peptides of a substantially similar amino acid sequence, at least one said peptide conjugated to the nanoparticles by means of a Cysteine (C) residue and wherein the nanoparticle conjugate further comprises a ligand attached to the peptide, wherein said Cysteine residue is located in a central region of the peptide, and/or wherein said peptide includes two adjacent Cysteine residues.
